(19) United States
(12) Patent Application Publication
(10) Pub. No.: US 2025/0279215 A1
(43) Pub. Date: Sep. 4, 2025

(54) Metadata mapping calculator forecasting personal distancing and environmental safety actions

(71) Applicant: Jeannie Marie Kozak, Columbia, MO (US)

(72) Inventor: Jeannie Marie Kozak, Columbia, MO (US)

(21) Appl. No.: 18/591,495

(22) Filed: Feb. 29, 2024

(51) Int. Cl. G16H 50/80; G06Q 50/26

(52) U.S. Cl. CPC G16H 50/80; G06Q 50/265

(57) ABSTRACT

Predictive modelling was demonstrated in software forecasting site-specific, user-specific recommendations for levels of physical distancing from novel airborne pathogen sources, and can forecast corrective safety actions mitigating other environmental hazards, and be combined with other environmental predictive safety models for economic full safety software package, with recommended corrective actions including social distances, safety actions, and control of the source, receptor or path in between, early recommendations and distancing found prevent future novel pandemics (Kaur 2021), and main initial calculation with highest degree of accuracy being social distancing and appropriate actions to protect against the early pandemic identification and preliminary transmissibility characterization studies, because the main use is outdoor calculation of safety actions since the modelling currently relies on meteorological data, but this invention would likewise predict mitigation actions and quantities anytime air or other environmental media behaves similarly.

### PRIORITY CLAIM

Predictive modelling has been demonstrated in a software application for mobile or personal computer which forecasts site-specific, user-specific recommendations for different levels of physical distancing from novel airborne pathogen sources, and it can also forecast corrective and/or safety actions to mitigate other environmental hazards, and be combined with other, existing environmental predictive safety models for an economic full safety software package, with the recommended corrective actions including social distances, safety actions, and/or control of the source, receptor, or path in between, with early safety actions and distancing found to be key to preventing future mass death from novel pandemics (Kaur 2021), the main initial calculation with the highest degree of accuracy will be social distancing and appropriate actions to maintain survival from the early pandemic identification and preliminary transmissibility characterization studies, because the main use is outdoor calculation of safety actions since the modelling currently relies on meteorological data, but this invention with modelling would likewise predict mitigation actions and quantities anytime the air or other environmental/ambient media behaves similarly.

### OVERVIEW

The current model calculates safety actions, namely social distancing for COVID-19 and similar airborne pathogens. The model currently bases the calculations on mass transfer and air transport of Particulate Matter (PM) that makes up transmissible airborne particles, as well as the significant exposure times and relative susceptibility of users thereto. Meteorological parameters are the main determinant of current safety calculations, which are currently developed for air transport of hazards, and to be developed for other environmental media. Unlike other similar recent inventions, especially in light of COVID-19, which rely on theoretical fluid dynamic modelling dependent on site-specific factors, or relatively well understood indoor ventilation engineering, the modelling for this invention, “software application with modelling,” currently consists of a simple scoring equation based on rating of factors from literature and preliminary studies along with weights for each factor.

This low-variable, universally simple multifactor weighting is the basic structure of an artificial neural network (ANN), so pilot studies with feedback about, for example, if the user got the disease or not could be used in an ANN to show if there is a significant increase in disease transmission without the calculated social distancing and/or other safety actions. Other new statistical tools to test to date include AI options similar to the ANN algorithm, and/or traditional process feedback equation (e.g. Monte Carlo simulation) to rank the weights and to correct the factors.

Meteorological metadata are used to calculate categorical ranges of physical distancing for established and novel hazards, with the options to input other metadata correlating with increased risk in that area, as well as user-specific data indicating susceptibility to each hazard. The updated modelling equations replaced the traditional industrial droplet evaporation model used by the Centers for Disease Control to obtain their “six-foot rule” with environmental regulatory modelling equations, and updated them with novel insights into environmental transport and transmission to end-users or organisms. As the main function is to forecast a spatiotemporal set of environmental safety actions, the coding can likewise use current and past meteorological metadata for current and past meteorological variables for any specified location, and use past outbreaks with IA or statistics to likewise screen variables, update transmissibility and susceptibility factors, and create data set libraries to train the IA for future outbreaks.

The model currently predicts extent of transport and transmissibility and outputs recommended physical distancing for the present moment and hourly for that location for the duration of the seven-day forecast. It can incorporate additional models and outputs for additional spatiotemporal hazard assessments. It includes daily summaries for morning, afternoon, and evening or overnight as available from the metadata source. The coding is currently written in JavaScript but would probably work better if recoded into a more metadata forward language such as Python, snippets of which are already freely shared with citizen scientists to upload their water quality metadata for public data quality assessment and mapping. Python coding is more geared to input of a wide variety of site-specific co-factors to be input as metadata and user-specific susceptibility. Modelling equations were originally based on the only fluid dynamic equations known to be used to develop the 6-foot social distancing rule, whose reference by the Center for Disease Control appears to have since been archived, and have since been updated for established empirical conditions, with plans to update as more insights are gained in hindsight of disasters, to be used for future novel ones (Chilton, 1952, Parts 1 and 2).

The factors used in this modelling were updated with these recent developments to assess airborne pathogen transmissibility (and later, other environmental hazards), combined and used to generate a score of transport and/or transmissibility and a set of distance ranges for such with our recent insights into environmental transport including transmission of novel biological hazards like COVID-19. Current and past data are also available to compare with predicted increased level of infection rates and measured environmental hazards, allowing additional means of testing, improving model accuracy and providing output metadata for use in government and private metadata mapping and quality control for data, sensors, monitoring, modelling, public understanding of these data and their limitations. Since this invention models environmental transport in a significantly simpler way, and since the traditional complex modelling of environmental regulation is headed out in way of a new generation of Low-Cost Sensors, other potential assessments from this calculator include source compliance for industry with environmental regulations. As the primary use for safety actions can be extended to compliance by the “source,” i.e. for individuals or organizations for distancing or requiring certain safety actions, this application can also be extended to industry compliance, contributions to risk, and local background concentrations of environmental hazards and transport thereof. The data quality of this new generation of public monitoring with LCSs is under global development while companies are being put out of business with their broadcasting on social media poor, false data from this new generation of commercial sensors, nearly all of which are still reliant on site-specific calibration with gold-standard traditional complex modelling by highly trained individuals and monitoring with costly equipment. This new AI-forward simpler model, which currently with its meteorological basis is equivalent to EPA's new petroleum fenceline monitoring background and transport equations in the regulation, also allows browsing and export of site-specific meteorological data for more holistic environmental troubleshooting to reduce consulting dependence and develop site-specific calibration parameters required for data quality from this new and growing generation of low-sensors for environmental assessment (ORD 2018, Williams et al 2018, Williams et al 2019, Duvall et al 2020).

With the concern of data quality, this application will include a distancing practice activity to reduce uncertainty from inter-user variability.

With source contributions to environmental risks classified as point source or area source for traditional pollutant transport modelling and monitoring, the same classifications can be used for this software application.

With a variety of recent similar products that indicate safety, none of them uses outdoor meteorological forecasting to predict the distance required to avoid transmission of novel airborne pathogens. However on Feb. 26, 2024 Microsoft launched point forecast mapping emphasizing commercialization of this GIS technology decades in the making.

The additional personal and local factors from the literature can be added with minor source code additions and allow more feedback for model testing and improvement. Aside from eventual machine learning, this tool uses simple existing equations validated for decades in real-world applications with simple source code development to provide a nearly free application for mobile or laptop use. This low cost will provide a barrier-free entry for lower socioeconomic classes into the Internet of Things for Health and Safety, upon which survival in the New Normal will continue to be increasingly reliant (Anderson et al 2021).

### References to This Invention Herein, All the Aliases

This software application and associated modelling will be referred to as “this invention,” “this product,” “this software application,” “this application with modelling and actuated automated output/safety actions” or combinations thereof for the remainder of this document, even though some of the equations remain external to the current coding. “This software application and modelling” also refers to this invention to emphasize the modelling. Both terms apply to the full embodiment of this invention; the aliases are used for emphasis and to not exhaust the reader with full description, while avoiding forgetting the overriding concepts with oversimplified terms.

### Naming of This Product

This product, consisting of at least two other product packages, consists of the software application, modelling and resulting actuated automated outputs described herein, with all these output actions umbrellaed as one else-named product. The names for the entire product and parts thereof have been tentatively designated as MetaSafe, MyMetaverse, MetaCraft, MetaMate, MetaGrid, MetaMine, and/or MetaversalESafe, with the social distance calculator portion of the product tentatively named as FarCast (Trademark Pending).

### Uses of This Product

This product is intended to be used personally on mobile phone or android/laptop computer and/or for population risks, with the metadata generated used for public advisories and model testing and validation, improvement, etc., but is not limited to either type of application.

With so many factors discovered to influence biological susceptibility to environmental hazards and exponential growth of machine learning, Internet of Things, and probable causes of environmental hazards and biological susceptibility to harm from them, this software application has the potential to unify the metaverse across the globe and beyond, for sustenance of human safety, which relies upon safety and sustenance of other ecosystems and life forms, which this software application may also provide a hazard index for as well.

### Summary of Methods of Software and Modelling Development

The method overview includes piping additional metadata into the source code equations just like the data from the NWS is currently doing, extracting variables and information, both quantitative and qualitative. Aside from eventual machine learning, this tool uses existing equations in this new application. The predictive modelling equations been tested for decades in the real-world industrial regulations and are continued to be validated and published to today, some for almost a century in industry. Recent insight from the COVID-19 pandemic have spurred additional simple equations used in the coding to predict relative degrees and actual ranges of distancing and other actions to ensure safety including biosafety (Coccia 2020, Bashir et al 2020, Zhao et al, 2020).

With minor, ready-to-use additions in coding like environmental modelling equations used daily for decades in industry, this software application has the potential to forecast a variety of biological-receptor-specific actions, some quantitative, to protect the receptor or user from a spectrum of environmental hazards including but not limited to airborne pathogens, airborne toxins, and spreading spills and leaks in any environmental media: air, land, and water, with the potential for space applications. For example, US EPA has just announced that their scientists have developed the Virtual Beach modeling tool to predict concentrations of E. coli, Enterococcus, and other bacterial microorganisms at both fresh and saltwater beaches (USEPA 2023 (2)). Modelling of surface, soil, and groundwater infiltration, transport and kinetics can likewise be incorporated into this metadata calculator to predict hazards from those media (Adeleke 2021).

The current focus of this development is transmissibility of novel airborne pathogens, with a forecast like existing specialized forecasts such as Air Quality Index and Pollen Forecasts, although this one is currently for the spatiotemporal resolutions from the primary source of model input data, the US National Weather Service. These input data currently have the strongest known correlation to biological transmissibility of airborne pathogens. Although a plethora of studies quickly emerged after the COVID-19 tragedy, we are still considered largely unprepared for the next ones. The current predominant predictive models are to be augmented with environmental release modelling that has been used regularly for regulatory purposes since the 1970s (Congress 1963), and these models can be updated with this software application and modelling described herein, which includes biological releases, replacing the only known engineering determination of the original COVID-19 six-foot-rule (Chilton, 1952, Parts 1 and 2). The structure of the equation is ready to add other factors as developed shown to dictate novel hazards from respiratory particle transport and kinetics (Arumuru 2020).

The output metadata can be mapped via packages such as ArcGIS at a spatiotemporal resolution provided by the incoming metadata, in the US, by the National Weather Service, and co-factored with a growing number of variables similarly freely available via open API data. Many of these co-factoring metadata have county-specific or greater resolution, such as geofenced polygonal weather alerts. All the modelling equations and code required for input and output sharing of these metadata through servers of the supplied and received metadata are established in literature and practice, and simple enough for minimal hurdles to implementation compared to similar predictive tools for personal environmental safety. For example, the Python code to export this software application's output data for water quality is openly available to citizen scientists to cut and paste into ArcGIS StoryMaps (USEPA 2021).

It currently can calculate social or physical distancing from characterized airborne hazards such as COVID-19 and air pollutants for an end-user's specific location, forecasted hour-by-hour for seven days with a daily summary for the morning, daytime, evening, and overnight like the weather forecast.

The distancing is then scaled by a user's susceptibility index to classify the distancing into three categories: low, medium, and high. “Low” is defined by the CDC as a 6-foot distance, although this distance was defined for well-ventilated indoor settings with a caveat of a 15-minute total exposure time (DiSalvatore et al 2023). Other factors that are currently input in other development as sensor data can be input by the user as expected conditions, as this software application is geared to forecasting and mapping recommended social distances and other environmental safety actions. These factors have been studies since the reaction to the COVID-19 pandemic (Ficetola and Rubolini 2020). Many share commonality with transport factors, such as volume of voice (the respiratory particles are ejected into the environment for longer-times in locations such as senior living facilities, bars, schools, etc. with longer lasting particles from louder and longer vocalizations such as in men, when singing, and where work warrants hearing protection) (Kopechek 2020).

Minor changes in source code can be used to develop a mobile software application that can map, for the present time and forecasted, the safest routes or precautionary or safety actions and quantities at any location, with the option to input an end-user's susceptibility data via user-friendly questionnaires.

During times of novel pandemics, epidemics, and toxic environmental hazards, planning outdoor activities is tantamount, and expected to be required for increasingly long periods of time while maintaining health, safety, and prosperity, especially for the lower socioeconomic classes. Factors for increased susceptibility for different socioeconomic classes, race and gender will also be included in embodiments (Pathak 2022).

### Source Code Using API Metadata Parsing and Output Metadata

The software application using this source code has the potential to use a full spectrum of interdisciplinary predictive modelling equations since the personal use requires consideration of user-specific data about each person's susceptibility to each hazard analyzed.

The source code currently uses open API data from the US National Weather Service for their hourly seven-day forecasts in spatial resolution of about 1.5 miles by 1.5 miles (2.5 km by 2.5 km). Probably the same resolution as Microsoft's Feb. 26, 2024 point forecast map advertisement launch on Windows' gateway to your Windows password.

The National Weather Service (NWS) API allows developers access to area and point meteorological forecasts, alerts, and observations, along with other weather data. This API is free to the public and offers a cache-friendly usage, allowing the mined data to expire once the software application is closed without requiring the source code to do it.

The API is available in several formats. The current source code extracts the API metadata in JSON format which according to the NWS promotes machine data discovery since JSON metadata is widely used and allows simple dissection of the variables used in the engineering and biological matrix of equations that are in the source code.

The output contains safety and precautionary actions and quantities for the end-users or receptors of the hazards and can continuously build more outputs as the metaverse expands and builds from everyone's discoveries into the future with more available application of AI packages to help the programs self-learn.

### Data Quality Assurance for Model Reliability and Reproducibility

The model is only as reliable as it is robust with reproducible results in different sites beyond calibration conditions. As learned in recent webinars from EPA about environmental regulatory data issues, broadly interdisciplinary checking by experts is required of the future of these types of metaverse software applications for them to be of actual use for public welfare and human and biological safety on this planet, for feasible habitation on large-scale biodome stations in space and on foreign planets, and other biosphere or planetary systems.

There are many emerging similar commercially available technologies claiming to help with social and safety issues, however, the users are usually unaware of the limitations of the data and need, as sited by EPA during these Low Cost Sensor Toolbox webinars, for site-specific calibration with gold standard environmental monitors.

For Data Quality Assurance of this invention, the same improvement in interdisciplinary checking is expected to be required. As these types of metaverse software applications become more and more prevalent, in order to be of actual use, there will be similar challenges in the growing field of metadata quality engineering. While the environmental compliance reporting and permitting used to be costly and burdensome to industry and handled on more of a case-by-case basis, now, this new generation of simplification and empowerment with technology can actually help industry by a good neighbor while showing their responsibility and even planning production and making decisions with the help of the new generation of metadata tools.

### Other Applications of This Invention

This “software application” (with modelling and actuated automated output actions) can be potentially used in any application where environmental data and transport/kinetics thereof are desired, especially for safety from hazards emanating from biological or other sources such as industry, portable sources, and other point and area sources of hazards. This invention can serve as a model to extend to other species of animal to safely inhabit ambient spaces or any space large enough to have air flow behaving more like outdoor climates with natural or controlled meteorology, rather than the exhaustively studied indoor air flow behavior that typically characterizes associated Heating, Ventilation and Air Conditioning (HVAC) capabilities as well as the ability to specify air filtration, disinfection, and occupancy better than has been achieved with outdoor ambient air fluid mechanics, although this study will attempt to cross classify such behaviors with the age old behavior of ambient air modelling for regulatory purposes.

The current application demonstrates one such example by calculating social distancing (i.e., physical distancing) for COVID-19. Traditional fluid mechanical equations are used along with the worst-case and best-case meteorological conditions for survival and transport of excreted respiratory droplets and aerosols, i.e., respiratory particulate matter (PM) (Carson et al 2021, Mecenas et al 2020, Peci et al 2019, Prata 2020, Qi 2020, Runkle 2020, Sarmadi 2021). These equations are similar to EPA's new fenceline monitoring corrective action-triggering equations using basic wind transport, and they were used to develop the model's “velocity factor” along with air regulatory experience for ranges of wind velocity resulting in either laminar or turbulent flow, and noting that PM travel velocity profile is directly equal to wind speed during laminar flow, while during turbulent flow, the PM travel is less than wind speed due to eddy diffusion and dissipation of the respiratory spray. However, for diseases with extremely low significant exposure time such as TB, and/or those transmissible from spray nuclei (evaporated spray), transport velocity profile is assumed to be equal to that of the of wind speed will be used to calculate potential distance travelled before significant dissipation is expected to occur.

The user could choose their own activities based on meteorology and their level of susceptibility to any number of pathogens, toxins, or allergens. The outdoor activities can be scheduled and locations chosen for a number of activities vital to health, especially during environmental and biohazardous emergencies, especially when they are novel and not well characterized. The lower socioeconomic levels can be better protected while allowing health and safety for such activities proven during the COVID-19 pandemic vital for human welfare, such as outdoor exercise, recreation, enjoyment of the arts, receiving free lunches in parks during summer, outdoor schooling and enrichment activities, even haircuts and finding affordable clothing and other household items especially for those relying on used items. Others may choose to avoid situations.

Food safety will also be sustained by this software application, as farming outdoors is a critical and threatened resource in the face of the age of pandemics and severe weather, with vegetable and fruit farming on smaller scales requiring more close contact. Small scale farms and a variety of fruits and vegetables will become more critical for food security for lower socioeconomic classes when supply chain issues persist into the future of this new normal.

First responders to medical and environmental emergencies will be able to determine distancing required and if masking is required for closer contact. Realizing that natural disasters and unlivable climates are the new normal, it will help not have further medical burden if smart masking and distancing can be practiced especially in emergency situations.

Numerous additional outdoor labor can use this software application that are expected to deploy during times of environmental and biological crises, such as construction, infrastructure, security, etc., while other labor may turn to this software when their services and goods production turns outdoors during initial phases of outbreaks, before better characterization and preventative measures such as vaccination are initiated. With temperatures rising many can not work or live with masking due to sweating rendering them useless.

For those who mask less than the general public for health or other reasons, this software application can help them choose daily life actions as alternatives to masking, or it can provide feedback metadata to provide evidence for masking or other precautionary measures that may or may not be being taken (Freckelton Qc I. 2020). These trends may be locationally based, and so can be compared with the exponentially growing available metadata databases. It may support keeping all these metadata freely available to the public, and may support and leverage investment in increasing metadata pipeline capacity and security.

As masking continues more health issues are discovered to be associated with it, including mental health in schools (Ayuso-Mateos et al 2020, Bhattacharya S. 2020). The decreased masking allowed by this software application would be particularly valuable to groups with increased health conditions adversary to masking, and those required to mask more extensively. Such groups include elderly especially living in senior facilities, mentally disabled, those with compromised breathing, those who exercise to manage health conditions, and school children. Again, there are additional uses for this software application by the socioeconomically disadvantaged populations. For example, the ability to school at home requires far more financial resources, flexible employment, and usually more than a single parent.

Along with the main use of social distancing calculation and other environmental safety actions, a major category of use for this invention is the evidence-based decision and risk assessment of avoiding masking and other safety restrictions. As resources and food security diminish, the good fishing spots will be more crowed and guarded than ever. Social outdoor exercise, sports, arts, and general health can continue. All activities are even more difficult when very hot and humid, whose severity is expected to accelerate in the face of power blackouts/brownouts and increased population density. The sweating during masking also renders them unreliable, so turning to outdoors and masking for novel pandemics and environmental threats is another dwindling option for which humankind remains largely unprepared, with the potential for mass death and suffering (Shafaati 2023). However, we have learned a new way of life with additional opportunities to prepare for this new way (Purdue University, 2020). As urban streets are increasingly serving as homes with less resources for sanitizing them, the airborne nuclei will also be more of a concern to avoid with calculated distancing and categorical ranges forecasted for locations for better survival and thriving planning.

This software application can accommodate current and future trends such as behavioral and mental health trends. For example, when more risky behaviors rise due to more unstable mental health, the user may use their own information about an environmental hazard not specifically calculated by this software, but they can assess any such specific risk with the software's elevated risk of spread of any hazard in the four environmental media categories (air, land, water, and space). For example, say the divorce rate and re-marrying rate rise, along with a quarantine habit developing in having workday sexual activity. Then, since fecal and sexual fluidic nuclei are more likely to become airborne with frequency of sexual activity and clothing trends, individuals may choose to avoid known sources (McDermott et al 2020). Other upcoming biological hazards from humans and other sources that would use the modelling herein could include skin shedding from dermal sources, for example a recent rise in leprosy in Florida, vomiting and diarrhea aerosols or nuclei along with fashion trends to not require covering source areas and with a large amount of disease for example food poisoning coming via FOT (fecal oral transfer), etc. including disease combined with climate threat, food insecurity etc. using this multifaceted modelling.

This software application can extend to any other environmental media, including space habitation. To ensure safety in space habitation, design of biodomes in space stations would need to model whether the fluid mechanics of the air behaves more like outdoor ambient air patterns or indoor air ventilation. In these environments human and animal safety will be tantamount to success and biological susceptibility.

### Use for Other Hazards, Modelling of Special Hazards with Additional Transmission Assessments

The use of this invention for other hazards and the modelling development thereof can extend with similar environmental engineering knowledge to the various environmental media as discussed. In depth assessments of airborne pathogen transmission will be the initial focus and the modelling of the other media will be adapted with future anticipated collaborations based on previous employment by and local colleagues of the inventor in the media of waste water treatment, ground/soil/surface water transport and kinetics in complex terrain such as karst, with the main expertise in air.

For the extended air studies, modelling of previous pathogens will first be incorporated and tested. Previous pathogens such as tuberculous as well as future, more contagious ones may have higher distancing and lower significant exposure times, especially in the face of increased environmental hazards and disasters and weakened immunity of biological organisms, both known to be exacerbated by our increasing population density, which is a metadata input for the social distance calculator (Martins-Filho 2021).

“Medium” and “high” categories are supported by increasing studies showing long-range transmission of airborne pathogens in well-ventilated areas. These categories are caused by meteorological conditions ideal for longer range transport of respiratory (or otherwise excreted and pathogenic) particles.

### Determination of Categorical Cutoffs

The “high” category would contain ideal co-factors signaled by metadata input openly available by similar API data from public entities such as the US EPA, US CDC and private organizations mapping citizen science data and other metadata such as ArcGIS (USEPA 2020 (3)).

### Source Coding

This invention can be featured as a widget, or used as the main application with other features coded in as widgets. There are in increasing number of such widgets publicly available as open API including EPA AirNow Air Quality Index and AirNow Fire and Smoke Map, which can be incorporated by this software application without cost, and both of which are being supported by lower cost air sensor calibrations (Barkjohn 2022).

### On Parallels to Exhaustive Environmental Regulatory Engineering, Don't Reinvent the Wheel

Air modelling of exposure mapping of ambient air plumes such as from industry or natural disasters has been exhaustively studied since its inception from the Clean Air Act in the 1970s for Permitting and Reporting of Major Sources of air pollution. These regulatory models are to be compared with the latest biological parameters of respiratory expiration measurements so that these same tools can be used for biological releases along with modelling invented as described herein. The models can be supplied to public entities for social welfare. They can also be compared to the other predictive models found in literature but not used to develop the modelling herein. These models were not used because they would require more testing and were more theoretical compared to the empirical factors influencing transmissibility. Additionally, the types of simpler equations such as those in the modelling herein that model the regulatory meteorologically based air PM transport and rely on Low Cost Sensors have been found to be too site specific to use most of these sensors reliably to date, but the meteorological basis is the same no matter what is measured, so the end result being “did they contract the disease” could be a model validity check. However, this data as discussed would have to be from user who can reliably estimate social distancing, perhaps with a co-application game type testing of and improving their estimations. Additional tools to improve modelling herein include Waste Water Treatment testing and epidemic metrics, vaccination rates, etc. as discussed. Other regulatory models to compare include TANKS for Leak Detection and Repair program, planned by the author to combine with drone technologies to check the leaks, and air monitoring available for applications such as fenceline monitoring (LIDAR, active sampling, and other exhaustively analyzed new technologies reducing need for expensive meteorological and ambient air monitoring and modelling used since the 1970s), MOVES and other portable source models, and other models to aid with environmental regulation such as the Diesel Emissions Reduction Act and allotment of resources according to modelling such as from the Volkswagen Trust. Even more ecological factors can enter the package such as nighttime light pollution, herbicide use and suspected effects thereof, species counting citizen science, etc. The variables of this study herein can also be tested such as anomalous atmospheric circulation seen from remote sensing and other technologies. Human factors such as deforestation versus suspected effects such as disease outbreak and immigration patterns are a couple of examples of the endless applications using this application described herein.

The current trend is to replace traditional burdensome compliance demonstration tools with a new generation of low-cost environmental sensors with improved modelling and data quality assurance. The data quality targets must also be determined. These tasks are using global expertise and technologies and have been concluded by EPA to require “a lot of grey hair.” This is another bonus of this technology as it can utilize greatly needed expertise in the face of increasing trends in age related unemployment. As more of the fenceline type monitoring replaces traditional subjective and even non-transparent regulatory enforcement, the lessons learned about the new generation of user-friendly modelling can be leveraged with this exact type of metadata Metaversal safety application. As these low-cost air sensors are often used but little is understood about the quality of their data generated, the implications of broadcasting false data by so called citizen scientists is hurting an already struggling global competitiveness by the same people who tout the need for US goods at fair prices. These data are used in most air biosafety applications to determine hazards, but the performance targets of them are still in development by the US EPA with help from experts around the globe, all needed for a global collaboration in developing data quality standard determination methods for various Low Cost Sensors to ensure air quality standards are actually met and not falsified (ORD 2018, Williams et al 2018, Williams et al 2019, Duvall et al 2020).

This software application is currently set up to input US NWS open API metadata, but could use input from a variety of other sources such as other countries' forecasting API data, as well as data for the same meteorological air variables that are currently mined from NWS via parsing in source code and extracted using the code, including but not limited to: temperature, relative humidity, wind speed, wind direction, chance of precipitation, and weather alerts (indicating atmospheric instability). This is a living dynamic system of data engineering that will require continuous improvement to adapt to our changing medical models and more importantly, like the crux of this software application, anticipating future safety demands.

### Use of Literature in Selecting Initial Factors (Beyond Meteorological)

The equations developed and used in the overall scoring equation contain primary factors concerning PM transport and kinetics, along with secondary and tertiary factors shown in the literature to correspond to transmissibility, with cofactors examined and removed so that a minimum of evidence-based factors remain. (The primary factors are the main determinant of transmissibility and use the same meteorological variables now heavily relied upon for compliance demonstration. As discussed, they usually allow weather station monitoring, NWS data, or a combination, and often gather air quality data from a new generation of fenceline monitoring regulations, for example for ambient Hazardous Air Pollutants (HAPs) such as for benzene from petroleum refineries.)

The secondary and tertiary factors come from published factors of airborne pathogen transmissibility and were assumed equal or ranked in weight separated by an initial, significant amount of at least 10%. They include latitude and longitude, season, regional degree of stability and type of general climate (maritime, hinterland, valley inversion, etc.) atmospheric circulation, and hyper local humidities (Balboni et al, 2020, Sajadi 2020, Sanchez-Lorenzo 2020).

With several potential embodiments factoring in variables studied in remote sensing of the environment and natural resources, even to predict social distancing for pandemics like COVID-19, as well as in precision technologies such as precision agriculture and precision medicine, future embodiments of this software application may be used to provide feedback for a variety of emerging precision technologies using remote sensing.

### Proactive Planning for Need; The Future is Now

Planning and risk assessment cost humans a large proportion of tax and insurance value and this is only expected to increase with climate and environmental hazards. This software application is expected to be used mostly for planning and risk assessment so its value in infrastructure and human civilization construction, repair, maintenance, and safety is expected to be one of an array of new precision tools using the metaverse and new sensor and data technology.

### Options to Test a More Elusive Factor: Biological Susceptibility

The option to input an end-user's susceptibility data via user-friendly questionnaires allows incorporation, testing and learning about medical models including gaining more evidence for automated medical decision-making using less published traditional medical models like Traditional Chinese Medicine which is increasing publication for evidence-based healing where Western medicine is increasingly failing to deliver. An obvious benefit is less taxation on our healthcare system and associated costs using taxpayer dollars.

### Widespread Use of Modelling and Metaverse Data Engineering

The engineering and biological equations used in the current source code comprise a simple yet highly interdisciplinary model which generates the output as actionable precautionary measures including but not limited to social distancing, Personal Protective Equipment (PPE), sheltering-in-place with various air filtering and other precautions, and choosing a safe evacuation route and time to safely travel.

### On Other Environmental Media

In all environmental media, point source releases follow plume behavior with dilution, and have been characterized based on airflow, groundwater flow, and surface water flow. The research and development of models often is often used for regulations, so there is much less characterization for non-regulatory purposes. However, the USEPA does study this non-regulatory category of potential environmental hazards to support regulations, technology, and anticipate future demands so development of corresponding supply can be done proactively and not reactively in haste as was repeatedly documented for the COVID-19 pandemic.

### Feedback

The concept of a biological releaser behaving similarly as other environmental releases will be developed with feedback from this software application output metadata. Pilot tests may be conducted around hospitals to determine the number of ensuing cases or trends thereof, with support proposed from Roy Blunt NextGen Precision Health Building in Columbia Missouri USA.

### Modelling Poised to Adapt to Rapidly Accelerating Metaversal Technologies

Humankind has managed to not go extinct for millions of years but recently numerous hazards of snowballing threats has put the ship on a possibly unescapable collision course, on which outdoor social existence now depends. Even if population is curbed, all possible technology is developed, all resources conserved, and extra-terrestrial habitation is managed to commence, the fate of humans is largely agreed to depend on accelerated mathematical tools such as those used by this invention and in fact what this invention is a tool to wield the necessary relationships found by sister technologies such as data quality assessment, data mining, Internet of Things, and data engineering.

The structure of the equations of modelling is that of an Artificial Neural Network with literature-based ranking of factors as an initial base (with meteorological factors being the obvious main driver, and that factor derived similarly to EPA's assumptions about pollutant transport for new fenceline monitoring-based compliance demonstration as discussed.)

### Models from Regulations, for Regulations

Regulatory technology leveraged and applied in this software, thus possibly checking its accuracy include the following: Portable source emission modelling, Environmental Justice tools and mapping (Kunzli et al, 2003) and the Deisel Emissions Reduction Act (DERA).

With current air quality and fire smoke now mapped by Google as part of its standard metadata, this software application is the logical extension to use in forecasting, mapping, and planning actions such as escape, distancing, sheltering and other protections.

### Model Plasticity from Machine Learning

The matrix of modelling equations comes from publications relating environmental transmission from source to receptor, but can easily expand to incorporate more findings as well as use simple engineering process parameter screening methods to discard or reduce weight of less significant variables and relationships. As such, it has potential for machine learning and testing a wide variety of factors potentially related to reception of airborne hazards and susceptibility to these hazards. It is adaptable since we are in a learning process without the need for highly trained mathematicians, although these experts can now adapt their skills to validating the large data sets that will be generated!

### Other Uses

The modelling matrix of equations used in this software application incorporates new insights into airborne pathogen transmissibility to forecast personal safety actions, along with traditional equations of transport phenomena. The safety actions may include but are not limited to social distancing for novel airborne pathogens of a distance calculated by this software application, sheltering-in-place with additional precautions such as type of air filters and Personal Protective Equipment to use, and evacuation with associated personal precautions along the evacuation route. This application determines the extent of transport of viable hazards based on the hazards' predominant and subdominant modes of transport and kinetics, and primarily by the meteorological conditions upon which these transport phenomena most strongly depend. As soon as the predominant and subdominant modes are characterized initially for novel hazards including biohazards, this model will enter the weighting constants which will score the transmissibility to provide distancing and other corrective action recommendations. Then, after the hazards (especially biohazards and airborne pathogens) have been classified for appropriate social distancing and other precautionary and safety actions by the appropriate authorities, this calculator can continue to recommend safest actions. Additional inputs to model will be added such as extent vaccinations have been implemented. Numerous citations agree that more precise tools would have saved numerous lives during the COVID-10 pandemic, and that the response to it was a string of reactive, disorganized, costly attempts to save lives instead of proactive planning (Alexander et al 2021).

### Model Development

While many applications exist to classify distancing indoors using data input from air sensors and monitors, this patent proposes to develop a user-specific, actionable classification for any novel airborne pathogen or other environmental hazard or biohazard. For airborne hazards and biohazards for example the minimal characterization to be done a prior for this software application and modelling is the initial transport studies, i.e. the estimated distribution of transport or transmissibility parameters such as relative transmission via (respiratory) droplets, aerosols or nuclei thereof.

The initial study of the initial spreading of COVID-10 in a restaurant in Wuhan, China highlighted the importance of laminar versus turbulent air flow in indoor ventilation systems with respect to transmissibility of airborne pathogens (Li et al 2020, Shereen et al 2020).

COVID-19 proved the long-held expectation by public scientists and industry alike that severe pandemics are our future, especially if they are influenced by the suspected anthropological changes, many of which increase exponentially with population growth that is not expected to slow. In fact, many families are having larger families as a current trend—both intentionally and beyond their family planning capabilities.

Environmental plume modelling has been used for decades to ensure public safety. The models have recently been updated to include more mapping, prediction, and data quality assurance. Recent explosions of sensor technology have spurred further insight to be used in this software application. The current focus of the research and development of the related tools provided by this software application is on air, but it extends to modelling and regulatory equations used for safety predictions for other environmental media. The equations for all such environmental media are routinely used by local, state and federal regulatory agencies. This modelling may extend to space exploration and biodome habitation.

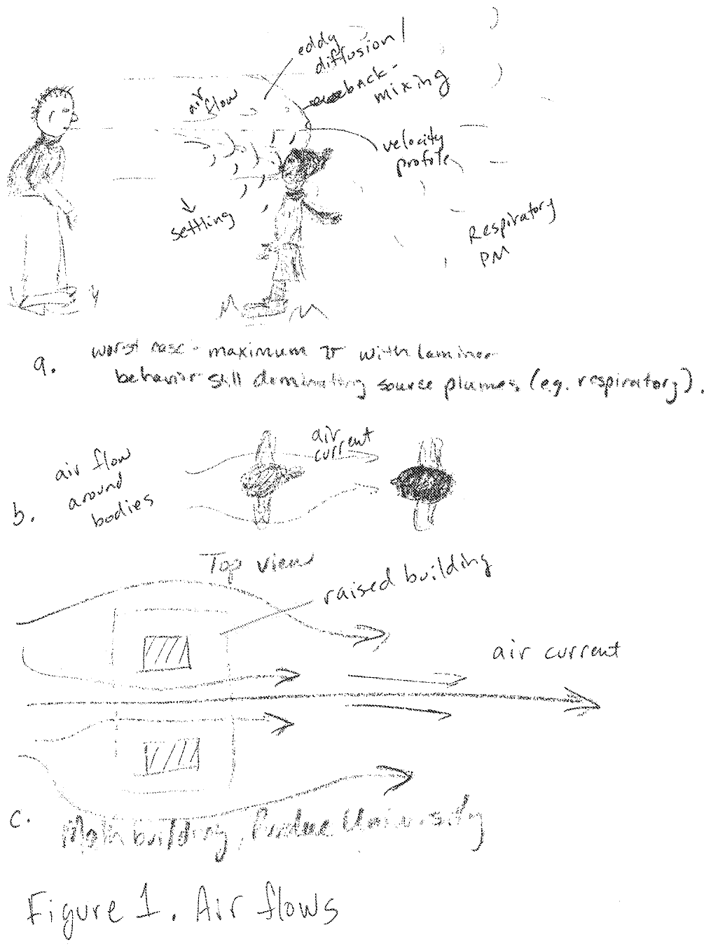

As shown in FIG. 1, the settling and transport characteristics along with evaporation and dissipation rates of basic plumes were used as the basis for transport and kinetic modelling of respiratory spray. Worst-case scenarios such as a “wind tunnel” effect seen in some buildings, and other air transport factors were used to develop cut-offs in ambient conditions. These cut-offs are highly specific to pathogen's significant exposure times. With the spray described in other sections, if exposure times are low or instantaneous and there is significant spray or even spit or nasal secretions ejected purposefully as is common in many cultures throughout the world and in the US especially where those culture are in population density flux, the habit of spitting or blowing one's nose onto the ground or floor can potentially cause sudden mass death given the uncertainty of emerging novel pathogens in the face of accelerating ecological global conditions.

Factors influencing the scoring generated by this software application's modelling primarily derive from physicochemical transport and transmissibility, therefore having significantly more weight than the more indirect factors referred to herein as secondary and tertiary, which will be discussed in more quantitative detail herein.

The more indirect factors derive from preliminary COVID-19 studies in the literature and were screened to eliminate potential codependent factors. They include the following.

### Prevailing Meteorological Trends such as Maritime, Inversion, etc. (Italy Article)

Wind temperature, speed, relative humidity including factors used initially by CDC to support original/initial development of their “six-foot rule” (later supported with clinical experience during medical exams of the six foot distance and significant exposure times). These weather data currently come from NWS, and can also come from country forecast API data, meteorological statistics, or climate influenced artificially or existing within biodomes on farms in long-term space stations. (They can be used to determine climate controlling factors in biodomes larger than can be characterized by indoor ventilation air transport.) NWS provides this API data for resolution to approximately 2.5 by 2.5-kilometer areas. Therefore, the distancing calculator is specific to this resolution of grid. The study on the first two waves of COVID-19 outbreak in Italy underscore the dependence on airborne respiratory excretion transport on the meteorological conditions plus the prevailing meteorological trends (Lolli et al 2020). While this was true for COVID, these factors will likely be of even more importance for outbreaks of more contagious pathogens (i.e. with low significant exposure times) such as was seen for tuberculosis, which is characterized by transmissibility via aerosols, or with the high weather-dependence seen with pneumonia, whereas COVID is believed to be spread more dominantly by droplets which are more resilient to evaporation, require higher significant exposure times, travel less deeply into lungs, and settle more rapidly. With the smaller aerosol particles and particle nuclei (solids remaining after a liquid particle evaporates) entering more deeply and settling more stably in the respiratory tract where the infection is initiated, these viruses have much lower contact times and greater social distancing required. Both types of air transport are well-characterized, used in industry since the 1950s, and were once claimed by the CDC as the basis of developing their six-foot rule for COVID-19 protection (Chilton 1952 Parts 1 and 2). However, they were cautious to publish that social distancing does not replace masking, which, once a more evidence-based social distance can be calculated from this invention, masks may be avoided, as indicated in numerous situations as discussed herein.

Population density—literature shows need for additional distancing and other safety actions with population density (Martins-Filho PR. 2021). Since this study emphasized the increase in death associated with population density in early stages of COVID-19, it may not seem obviously associated with a need for increased social distancing. However, the factor was chosen for increased social distancing need because the data from this study did indicate that the increase in mortality occurred despite safety actions taken. There are a few hypotheses for increased social distances with population density. One is that while we think we are socially distancing, in reality, in more crowded situations the distance is often not kept. Other factors include differences in air parcel behavior and higher ambient load of pathogen, and possibly lower health in more crowded populations (Ericsson 2024).

Air Quality—Biden's Environmental Justice Mapping Tools using Trump's increased monitoring to replace burdensome Permitting and Reporting via his Red Tape Reduction Program (FR 2017) as discussed, and these metadata are examples of the growing metadata engineering and technology, allowing exponential economical/resource leveraging and cross-checking one another.

Socioeconomic Status—Biden's Environmental Justice Mapping Tools. This app can help individuals inflicted with homeless, imprisonment and other institutional needs by reducing need for masking and allowing safer sleeping and other care for workers too.

Surrogates for factors increasing transmissibility (to be tested) include mask compliance and compliance with other health precautions such as existing county-level metadata like vaccination rates, breast feeding rates, and other indicators of precautionary behavior like helmet laws, seatbelt usage rates (by US county in downloadable file, Wikipedia), DWI rates, COVID rates and susceptibility factors such as race, age, disease, many of which are also mapped on county-levels and with higher resolution mapping, such as through Biden's Environmental Justice mapping and via ArcGIS metadata mapping of continuously more metadata, many of which are now supplied by citizen scientists and reviewed for data quality control, which is actively improving and being studied by groups such as the USEPA (ORD 2018, Williams et al 2018, Williams et al 2020, Duvall et al 2019). As discussed, these metadata can be used in partnership with other environmental regulators. Environmental markers, such as checking if processing plants are achieve the growing goal of zero discharge could be assessed with this software as it is a user-friendly browsable, cacheable weather app to use in industrial planning of operations with environmental consequences (Okos et al 1996). Established surrogates for pandemic level include waste water treatment plant indicators.

In summary, the driving force for environmental transport is meteorological and environmental quality metadata. Increasingly humans are finding more relationships between transmissibility and exposure to increasing co-factors available as spatiotemporal metadata. Everything that affects how we get sick and injured from environmental hazards is widely interdisciplinary, requiring interdisciplinary biostatistics, coding, and engineering, yet with ranking of studies in literature and knowledge of environmental modelling, it's the human checking and Data Quality Assurance that need the experience of once-employed environmental regulators and consultants, now serving as experts as automation of the process industry had to replace technicians and process engineers during approximately the 1990s to 2010s, using expertise from them and adapting to more automation expertise needed in our global competitive future.

### Need to Share This Information, Social Justice

While many thought that outdoor activities provided safety from airborne hazards such as COVID-19, several super spreading events have suggested the contrary, along with Trump's warning that COVID-19, with all its millions of fatalities, is “nothing” compare to expected future scenarios.

From a recent study by the PEW Research Center, “A plurality of experts think sweeping societal change will make life worse for most people as greater inequality, rising authoritarianism and rampant misinformation take hold in the wake of the COVID-19 outbreak. Still, a portion believe life will be better in a ‘tele-everything’ world where workplaces, health care and social activity improve” (Anderson, 2021). This software application is the exact Internet of Things product to fill this niche, ensuring a more equitable future for historically disadvantaged populations, improving the digital divide by the extreme low-cost and simplicity of the equations and source code used, especially compared to the other similar products that rely on air sensors (that do not work well) (ORD 2018, Williams et al 2018, Williams et al 2020, Duvall et al 2019).

### Methods Used to Develop This Invention

The methods used to develop this software application with modelling and actuation based on safety categories are as follows. An application has been coded to use NWS variables in air transport equations. Other environmental and personal input factors will be added along with a geolocation feature so the user can generate a personal forecast for any location and current or future time within the meteorological forecasting period. There are several additional metavariables available through NWS such as natural disaster warnings that will be tested to determine their potential influence in distancing scores in pilot studies.

NWS Endpoints for Warnings include county-level and smaller geographical areas, defined with a polygon whose corners are specified with latitude and longitude geological coordinates: Severe Thunderstorm Warning, Tornado Warning Flash Flood Warning, Special Marine Warning, Snow Squall Warning, Dust Storm Warning, Dust Storm Advisory, and Extreme Wind Warning.

The scoring by this application and the coding used to generate the actions have been demonstrated for air transmissibility of novel airborne pathogens. Variables found to be related to community transmission of airborne pathogens include meteorological conditions, wind speed, temperature, and relative humidity (Feng 2020, Peci et al 2019, Prata 2020, Wang et al 2021, Wu et al 2020).

The six-foot rule is under scrutiny indoors and the reference to where the CDC found a basis to declare it has been archived (Isaacs-Thomas, 2020). While six feet has been practically shown to suffice for clinical medical settings, noting that often masks were worn, outdoors, science is suspecting the social distancing could be more like 20-27 ft under certain conditions (Bourouiba 2020).

Experimental studies to update airborne transmission model of this embodiment can include measuring increase in particulate matter from human plumes. Since PM10 is the assumed size of respiratory droplets, the volume of droplet is known. However, while several studies have attempted to predict transport with natural laws of evaporation and settling, this invention is for outdoor use and the uncertainty is currently too high for theoretical modelling. Empirical modelling was therefore chosen to result in a score and only three categories of recommended safety actions or amount of distancing.

While the current equations to predict exact ranges of outdoor distancing for COVID-19 are based on empirical models listed in the scoring sections herein, the empirical modelling equation is to be used to check other similar predictive exposure models such as industrial environmental regulatory models to adapt them for protective actions from airborne biohazards and other environmental hazards.

### Testing the Modelling and Incorporating Regulatory Environmental Transport Models

The simple USEPA air model for particulate matter (PM) is AERSCREEN (TCEQ 2019). It runs instantly and can be used to screen inputs for relationships. To adapt it from industrial to human pollution type sources, the AERSCREEN inputs would be adapted, for example, with worst case scenarios for human transmission: a maximum expected height, horizontal (vent) emission, velocity for example 100 mph or found in respiratory studies, downwash tested for example with local studies such as St. Louis, MO USA study of other airborne particles characterizing urban downwash.

Physical tests may start with maximum transport trials of dyed emitted particles, aerosol and nuclei. Sprays may be emitted by any method similar to respiration superspreading events, such as by but not limited to spray bottles and spray from mouth (Aschwanden 2020). Verify wind speed and meteorological variables with air sensors. Turbulent fans, laminar flow hoods, and high output hair dryers may initially serve to prove how far these PM can travel, especially for the cases where aerosols are thought to be the transmission route, as for tuberculous, where significant exposure times are extremely short, possibly instantaneous, such as for novel pathogens. With both smaller PM from air pollution and from viral airborne particles, these types of airborne particles are especially dangerous since they lodge further into the lungs.

Modelling is currently based on environmental transport conditions known to be either significantly higher or lower than average in transmissibility of pathogens and exposure to toxins. They are to be used to adapt environmental regulatory modelling equations to specific pathogens and hazards of concern at that current time and better yet as anticipated. This invention aims to predict actions before the start to be pandemic or otherwise devastating, especially for lower socioeconomic classes who are less equipped to be reactive to disasters.

### Future Options to Improve, Adapt and Test Modelling

Modelling is currently based on empirical conditions shown in literature to favor COVID-19, influenza, and other pathogens and environmental toxins. Since everything changes every day, and technology has tended to react in hindsight of the COVID-19 pandemic, there are ample weighting factors to sort meteorological and environmental forecasting and mapping of relative hazards and recommended physical distancing into these three categories of low, medium, and high. After the categories are created, they are scaled to maximum known protection needed (Arumuru 1994, Bourouiba 2020). For example, the minimum and maximum distances respiratory secretions can travel set the lower and highest distances for social distancing for pathogens transmitting primarily via droplets, such as COVID-19; primarily via aerosols, such as tuberculous; primarily via nuclei; and/or primarily via a combination of these transport media.

The industrial transport equations governing evaporation and transport of droplets, namely Raoult's purportedly used by the US CDC to develop the “six-foot rule” are to be compared to the other modelling equations used, to check the six-foot rule and to adapt these industrial equations to current and future anticipated threats (Chilton, 1952 Parts 1 and 2).

### More On Scoring, Categorizing Social Distancing and Other Actions

For scoring of the current embodiment example of this software application, published empirical relationships were used to estimate factors correlating to transmissibility of COVID-19, and in some cases influenza and/or other airborne pathogens. It generates social distance ranges for the general user and user with increased susceptibility. After rating the distancing as low, medium or high, the rating is scaled between the lowest and highest known safe social distancing outdoors, and thus a range of acceptable distances are supplied for a location for the 7-day forecast, hourly and daily summaries. The metadata parsed from the NWS are extracted by the JavaScript code and used in calculations. The score reflects the maximum transport in the associated significant exposure time. The coding currently generates the input factors for the equations to be used to generate the safety actions and quantities and will be incorporated into coding for a complete plug- and-play software application that allows geolocation and user-specified locations, as well as which type of forecast, possibly integrated with other spatiotemporal mapping and forecasting platforms.

Equations and air modelling were established via published literature, some of which was used by EPA to develop the “6-foot rule” that has been a standard in chemical process engineering for many decades, involving evaporation rates of droplets in cooling towers. This application may be used in any smartphone, android device, or computer to determine safe social distance for the effective (contagious) transmission of any airborne pathogen. The distance is dependent on and calculated from environmental, site-specific, real-time factors such as meteorology, air pollution and aerosol and droplet transport phenomena. The application has been coded with three separate apps to determine site-specific meteorological conditions and forecast. Other data from EPA's growing StoryMaps that have been shown in literature to affect airborne pathogen transmissibility can be incorporated, such as population density, air quality, average age or relative age groups, socio economic factors such as those highlighted in Biden's Environmental Justice mapping, and more as they are discovered (Executive Office of the President 2023, Martins-Filho 2021). The output would be a score that is relative to the social/physical distance recommended to be considered biologically safe, or in other words to achieve biosafety from an airborne pathogen standpoint. The range of scores would be divided into categories similar to those used by EPA to show relative biosafety and what range of physical distance would be deemed safe at the user's conditions. It would also show distances for more compromised users. The factors of each disease would include whether that specific pathology is transmitted primarily through aerosol or droplets, or both. The weather data used comes from the National Weather Service, who was at time of app creation updating their API data infrastructure to accommodate a large increase in real-time demand for these metadata. They change the variables offered from time to time and are open to suggestion.

### Variable Screening Methods

Later design stages will include screening of variables to ensure the best few are chosen to determine social distance and other safety actions. Variables will be screened with feedback based on number of cases to refine the significant variables. Simple traditional process engineering methods such as Monte Carlo variable parameter screening can be used initially with worst-case and best-case scenarios to use in the Monte Carol matrix.

Another way this application can be developed quickly and economically using existing technology will be to test the growing number of statistical tools commercially available. Newly commercially available AI packages will be used to test various methods of variable weighting such as Fuzzy Logic and Artificial Neural Networks, as well as multivariate analysis available through older packages such as SAS (SAS Institute 1987) to determine the best relative weights of input variables on outcome (hazard classification and actionable recommendations such as increased distancing or sheltering) and then test and build the data library such as for analytical equipment measuring a large number of variables, some related, some not, such as has been traditionally used to develop industrial spectrophotometry applications (Rohrbach and McClure 1978). More and more applications are refined using even the speckle or noise within the signal to find the more subtle differences among a myriad of input variables and complex, highly non-linear relations found in biological systems, technology without which further development and management of our human species could not exist.

### Application Testing

Application is to be tested in pilot programs in concert with existing air quality portable sensor programs such as those recently launched in Kansas City, Missouri USA to assess the data quality of portable air quality analyzers (Kimbrough 2018). Test outside of hospitals predicting on certain days to recommend increased social distancing and check if cases by hospital patients over the next 40 days increases significantly. One such hospital poised for local study is the Roy Blunt NextGen Precision Health Building in Columbia, Missouri, USA.

### Counter-Leveraged Emerging Technologies for Bio-Security

Summary of counter-leveraged emerging technologies potentially inputting data and used in conjunction with this software application include those using metadata for safety or security. They may leverage resources put into developing all sides of the technology, this metaverse software application, the input metadata sources, and the receiving platforms of the metadata (usually will be the same as the platforms for the input metadata source mapping). The potential conjunction technologies include the following: swarm meteorological sensors, low-cost air quality sensor technologies such as Sensor Pods or “s-pods”, active sampling, particle counters, as well as animal models for airborne pathogen transmission—laminar flow hoods already ensure laminar flow (assuming movement of animals characterized with respect to air movement—which has been characterized since the early COVID-19 pandemic) (Barkjohn 2022, Ericsson 2024, USEPA 2018 (1)-(3), USEPA 2020 (1) and (2), USEPA 2023 (1)). These will be discussed as applicable to recent regulatory developments.

In addition to this environmental self-training modelling for biosafety, other novel and developing air technologies offering industries the ability to operate more economically due to the current trend to require more environmental monitoring, such as the fenceline air monitoring, while requiring less red tape that has traditionally burdened US industries, thereby decreasing global competitiveness or competition with others less fortunate in their permitting process for whatever reason, lack of planning resources, opaque case-by-case judgements, etc. The additional federal air regulations for refineries to monitor benzene at their fenceline has incentivized development of an increasing number of economically-feasible technologies that are actively assessed by the USEPA (Williams et al 2020) and has opened doors for similar trends such as this smart social distance and safety action calculator. This software application can help defend industry against citizen claims, which often rely on unreliable low-cost air sensors and not knowing the limitations of the data. It can show that background toxin concentration generated from off-site sources by receiving air quality metadata ad by allowing browsing of NWS JSON metadata to check for times when the increased ambient pollutant levels could be from other sources or have other anomalies in the claimants' claims. (See file containing example output of JSON complete weather forecast hourly data for seven days for a specific point location, complete with weather variables, as well as the current concept-demonstrating app output showing a forecast summary associated with that JSON file.) It can also help to calibrate the low-cost air sensors with historic NWS data, or forecast action to avoid industrial releases, for industry to take corrective action or plan otherwise, and for defense litigation to help substantiate data for both sides of the event, the releaser and the receiver of the environmental or biological hazards.

This software application may be used to forecast industrial operations in the face of the Trump Administration's Red Tape Reduction Program (Office of the President, 2017). Shortly after the Trump administration took office, an Executive Order was issued to reduce the burden of regulations while not increasing costs of the resulting new work to be done. Numerous immediate memos to the U.S. EPA ordered an immediate launch of these efforts, followed by an Executive Order for an additional Enforcement Program for them, even down to scrutinizing which pollution enforcement cases may go to court and scrutiny of the process for determining such (Office of the President, 2017). These memos were submitted to the U.S. Environmental Protection Agency to find ways to provide more flexibility for and therefore economic competitiveness to federally-regulated industrial sources, thus ensuring scientific validity of restrictions and reducing unnecessary red tape. The overriding goal of these immediate Executive Orders was to “increase permitting certainty” for all regulated industrial waste media—air, water, and solid pollution, each with their respective compliance demonstration arms of permitting, reporting, and enforcing—improvements in all!

This new normal of environmental technology for compliance demonstration can go hand-in-hand with this invention, as it is a software application that can compare forecasts with environmental drift, and can thereby provide economic advantage for this new normal of environmental justice: using sensors and technology instead of burdensome paperwork and complex modelling requiring larger consulting firms. This is all achieved by free sharing of NWS point/area meteorological data available for browsing, as this “software application with modelling” is essentially a weather app with novel environmental modelling based on evolving studies and accelerating AI capabilities, and experienced humans for the next generation of top engineering experts to bring their interdisciplinary insights into this critical development field of data quality engineering.

Air modelling has been used for US air pollution permitting since the 1970s and is continued to be used daily by air pollution permitting agencies in every state in the US (Congress 1963). There are various levels and types of modelling such as large source detailed modelling, mobile source modelling, area source modelling and point source modelling, the last of these being available in detailed and in quick screening formats (TCEQ 2019). These models focus on air transport of characterized pollutants and can incorporate chemical kinetics dependent on co-factors such as meteorological data that is put into the models. USEPA'S AERSCREEN is a quick and easy way to characterize horizontal releases of air pollutants like respiratory exhalation and is examined to be used as a surrogate for such.

Technology is increasing at an exponential rate. As research is published, applications extend with same modelling to protect against chemical and biological warfare attacks, mass toxic releases from increased severe weather events, all while the Pandemic Age and age of other health related compromises threaten humans from increasing angles as suffered by many during COVID-19, more severe flu outcomes, and hazards in all environmental media expected to increase, including simultaneous increased threats to food security, ecosystems, and human habitat. New problems are creating new fields of study that usually being biological in nature require large amounts of data handling, mathematic devotion, and ability to communicate them within broadly interdisciplinary teams.

### Data Use, Quality and Automation

Metadata generated can be sent back through API servers to assist with statistical analysis of similar metadata used as inputs in the modelling equation systems.

Metadata can be shared on mapping/summarizing/analyzing platforms through the various agency servers. As with other related metadata for the user decision making, the options grow every day, so they would be tailored to current and predicted hazards of interest. Examples include metadata from the CDC (e.g., regarding spread of disease or predictors thereof) from NWS and EPA concerning metadata including Air Quality mapping at both large and small scales (for example leaks and spills), Waste Water Treatment Plant indicators of disease outbreak and the like.

### PLC Controllers for Automated Actuations

Air Quality metadata can be used with simple feedback control schema including but not limited to Programmable Logic Controllers (PLCs) using simple feedback control schema or commercial AI packages, or other control schema to automate a set of actuations to help protect the public. As discussed this could extend to help automate industrial operations to improve air quality and environmental quality.

Programmable logic control technology using proportional integral derivative multivariate analysis will be tested for the most robust signal or other indicator of transmission of environmental hazard, shown in biological systems to exist in the noise (Cheng et al 2003).

These control schemas will be used to test and improve the calculation algorithms, allowing for machine learning and building of data libraries similar to those commonly used in biological signal processing (m sorter, and to control climates in biodomes and on earth when outdoor large scale climate control becomes more mainstream and critical for habitation.

### Actuations Based on Established Engineering, Not Reinventing the Wheel

The scoring, social distancing and other safety actions are based on established engineering practices used every day for a half century in the public entities including, environmental regulatory, energy, waste, and natural resource fields. They have been improved by adding additional factors from literature regarding transmissibility of airborne pathogens as a first step in adding other safety factors. With scoring to range from least to most social distance or other safety action, this scoring of factors multiplied by weights results in a simpler equation based on evidence without having to reinvent the wheel.

The scoring will start with inputs of meteorological data, air pollution data, location, date, time, airborne pathogen of interest (with plug-ins for each pathogen as they become characterized and epidemic). The scoring will result in an output of safety actions. The current base of this invention is to output the minimum safe distance to keep from front surface of people.

Dispersion modelling and chemical engineering relationships will be used for air mass, heat, and momentum transport to score transport factors for droplets, aerosols, and PM to which airborne pathogens/viral particles can viably attach. Eventually, modelling will be tested and compared to the more theoretical equations developed in the literature shortly after the COVID-19 pandemic declaration. These equations were not incorporated into this modelling because they appeared to characterize minutia in viral transport using theoretical formulae that did not lend themselves in structure to this factored, ranked, weighted, and thereby categorized approach. Additionally the structure was too complicated to test in a variety of statistical options from traditional to new AI packages.

The inputs will come from geolocation and NWS or other weather source, then from other entities for additional factors to be tested (i.e. the more indirect secondary and tertiary factors from literature versus the robust weather variables dictating PM spray transport and kinetics). The existing meteorological and air pollutant data will be inputted, along with user inputs, location, and date/time.

Output data can be for a point location or county-wide, since many metadata co-factors freely available are county-specific. Embodiments can extend to other nations and beyond terrestrial habitation especially if metrological data are available, or surrogates thereof in the case of habitation in biodomes, space stations, and anywhere the air behaves more meteorologically to incorporate this modelling as opposed to the already well-exhausted ventilation studies and inventions using air sensors and monitors. One such limitation not mentioned in those inventions is the inaccuracy of the multitude of low-cost sensors relied upon.

This software application may be used to train other nodes such as those low-cost sensors if the end effects of physical distancing and other safety actions are used to demonstrate exposure assumed in those models. Another end-product example related to the current social distance mapping forecasting calculator is to use the same equations and code to output a “COVID-FORECAST and NOWCAST” for each county and major city (similar to the current AirNow ozone and air quality mapped forecast in categorical ranges of Green, Yellow, and Red), using similar methodology that the USEPA used for AirNow.

The scoring weights worst-case scenarios as practical worst cases. The model is currently based on established relationships for droplet transport dynamics and meteorological and other influences on virus transmission via droplets, aerosols, and nuclei. Add other suspected novel relationships will be added, not so much for the user, but to examine the predicted rates of transmission or exposure and use this end result to update this and other metadata-using models. For example, insolation from the sun could mitigate virus by UV but the mathematical relationship is unknown, so, in a simple Monte Carlo evaluation for example, the areas with maximum and minimum insolation and other factors as similar as possible can see if the exposure is significantly lower in areas, especially in smaller areas with more receivers of hazards and even more hazardous conditions warranting increased distancing, such as playground and school, prisons, church gatherings with singing, etc.

The hypothesis on which the model began was that droplets act like PM10 and aerosol, like PM2.5. Since the first studies for novel airborne pathogens characterize whether the novel pathogen spreads primarily via droplet, aerosol, and/or nuclei, this initial information will be plugged into the model to calculate social distancing mapping forecasting based on transport phenomena of the transmitted particles. Furthermore, significant exposure times to novel airborne pathogens, indoors or outdoors, is based off this characterization of primary, secondary, tertiary, etc. transmission mode, so once that is characterized, the exposure time is a function of that transmission mode so the model can reliably output exposure time for given distancing without reinventing the wheel.

### Future Studies of This Embodiment

Future studies of this embodiment can include other models to compare and use results in related designs to make them more robust. Any data that relates to respiratory PM evaporation, for example, and therefore transmitted, not so much for COVID-19, but to be proactively prepared for future diseases that are transmitted via respiratory nuclei. They include indoor models of air flow and quality control, especially to minimize disease transmission, modelling used in other environmental testing, nasal rangers, air pollutant kinetics and transport such as from portable sources or of more elusive pollutants such as ozone, oxides of sulfur, etc., alternate models of weather forecasting, modelling to develop surrogates for “insolation” to show relationships with evaporation of airborne respiratory spray, and insolation as a surrogate for heat inputs to simplify ozone predictions and make them more robust.

Eventually testing should be done for these more complex relationships with multivariate analysis as is often needed for the more complex modelling of biological system, which often have so many variables that the signal is actually only found in the noise within (Cheng et al, 2003). For example, with increased insolation, UV potential for viral destruction increases, but evaporation of respiratory liquid particles also increases which, if virus is transmitted primarily via aerosols and/or nuclei, the effects could be negated. Testing and feedback, especially with emerging technologies is planned. For example, new sensors, swarm (nano) robotic technology and machine learning, such as artificial intelligence and artificial neural networks, would also be able to be used to obtain data for use when testing with this modelling and software application.

In summary, this software application has been developed using National Weather Service API metadata to classify safe social/physical distancing or need to shelter or take other precautions for any airborne pathogen or hazard. It will incorporate more metadata shown in literature and other developments.

### Modelling and Equation Development

The equation currently used to forecast an appropriate social distancing for any point in the US or beyond if meteorological data are available is as follows.

The predictive modelling is based on published literature and guidelines from environmental regulations. The confidence in transmissibility will be ranked by a significant amount, or assumed to be equal to other factor(s), based on literature, transport phenomena of liquid and solid PM and traditional engineering judgement used by public and private entities to make regulatory decisions affecting US industry for half of a century. While the equations to predict distancing and other actions will change as understanding continues to develop, the idea is to predict these actions and distances for any novel hazard based on understanding of similar transmission and equations governing transport phenomena of environmental contaminants.

The current embodiment applies to novel airborne pathogens and hazards such as hazardous air pollutant (HAP) violations and accidental releases, but will be extended to include spatiotemporal hazards from the other environmental media including extraterrestrial habitation or anywhere the environment exhibits its typical behavior in the various media. For air, this embodiment covers outdoor behavior which occurs in semi-indoor/outdoor structures now popular for socializing. The garage door cafés can also be tested for optimal design. One idea is to use a fire for gathering, as the fire pushes air away and sterilizes it, while the shotgun design of many “holes in the wall” small businesses offer a laminar air flow opportunity inside with a fire table near the garage type door for the next generation of safe socializing.

The modelling will yield scoring to determine hierarchies of actions necessary to control spread of novel pathogens, especially as the global population peaks as expected by 2050 to 2100 and survival risk and evidence-based decision-making become life-or-death matter for entire families and societies. As many try to join the trend to turn back to sustainable family farming, hunting and gathering on larger plots of land with larger families, these actions will help level the playing ground for non-land owners who cannot afford these securities: food security, health and housing security, and safety. As acquiring the outdoor resources will require social distancing and other safety actions, this software application with modelling and automated safety actuations will help achieve “justice for all” be helping the less fortunate have equal chance for resource allocation. To get that one lucky fishing spot, hunting stand, or other honey hole, which if often not possible for the working class, which seems to be dwindling for wealth concentration, making this invention a tool for environmental justice and safety.

These equations are therefore aimed for survival in workplace, institutional, urban and suburban settings where masking is not always possible or in addition to masking. So, the equations will first predict if an outbreak is occurring by checking a Doubling Factor which simply assesses if the growth of the number of cases is increasing exponentially. If so, the spatiotemporal forecast for the distancing and other safety actions will be calculated based on a score from metadata.

### Early Epidemic/Pandemic Identification and Model Implementation

If the doubling score indicates epidemic or pandemic, these actions must be quickly implemented to avoid mass death as seen with COVID-19 (ECDC 2020). These actions will accompany certain embodiments of this invention. One such automatic action could be an automated raising of a precautionary flag similar to beach closing flags. Others could be indicators in factory settings, other workplaces, institutions such as schools and correctional facilities, hospitals, etc., especially since this invention is geared towards outdoor and semi-outdoor environments and to aid with Environmental Justice. Users can choose to receive notifications on a variety of devices on a regular or as-needed bases, similar to the US EPA's AirNow and beach E. coli forecast mapper released during the writing of this specification (US EPA, 2024).

The overall empirical equation (Eq. 1) was developed after examining the theoretical equations used to develop and verify the six-foot rule as well as AERSCREEN for industrial pollutant releases. Outdoor air is well-studied in industry, namely natural draught cooling towers, and in industrial regulatory compliance, namely air pollution engineering, meteorological modelling, and ambient air monitoring.

However, the overall empirical equation (Eq. 1) was ultimately derived from early COVID-19 characterization studies relating the transmissible distance of this novel pathogen to meteorological variables since they are the main determinant of PM air transport. With the high number of covariables and human interference in controlling experimental conditions, the published most and least favorable ranges of meteorological variables and conditions were used to scale the largest known extend of necessary distancing to calculate an exact distance, or score, and then sort it into three categories, each corresponding to a minimum recommended physical distance or other action such as avoiding certain events or sheltering with or without additional controls such as air filtration and sealing air leaks.

The overall empirical equation (Eq. 1) generates a recommended minimum social distancing for COVID-19 from a minimum of six feet to a maximum of 27 feet (Bourouiba 2020), and then to group this distance into one of three categories, each with its own minimum safe social distance. While effects of lockdown did not appear to be significant a wind velocity in the lowest measurable range of 1 to 3 mph did show significant increase in doubling rate of number of cases during the first 6 weeks of the COVID-19 pandemic, with additional increases in infection rate in area with poorer air quality (Coccia et al 2020). While masking, 6-foot distancing, and improved hygiene did show effective in infection rates, attention to meteorological conditions was shown to be recommended to prevent future catastrophizes, particularly low wind speeds which carry droplets without eddy or backflow diffusion and dilution of respiratory particles (Bashir et al 2020, Zhao et a 2020).

For Equation (1), temperature effects were empirically grouped into low, medium and high transmission, each associated with a range of temperatures while controlling effects of relative humidity (Bashir et al 2020). In Naples, Italy, during the first 6 weeks of COVID-19 pandemic, a significant doubling time of new cases was found to correlate with population density and air quality and whether the geographic location was near the coast, in an atmospheric pressure inversion (valley region), or classified as hinterland (Coccia et al 2020). While the population density likely doesn't play in actual transmission distances provided the user is social distancing, air quality and atmospheric stability have further support for transport. The infection characteristics were found to linearly correlate with these variables. This along with exhaustively published correlation of infection characteristics increasing with certain ranges of wind speed, temperature and humidity led to the following modelling equation to predict the recommended physical distancing:

Dₘᵢₙ*w_{SF}SF*w_{PF}PF*w_{DF}DF*w_{AQF}AQF*w_{VF}VF*w_{MF}MF*w_{OF}OF*w_{CF}CF=Dₘₐₓ Eq. (1)

where Dₘᵢₙ and Dₘₐₓ are the minimum and maximum known social distances for a given pathogen; SF is the Susceptibility Factor which currently is set to either “on” or “off” by the user with the User's Guide; PF is the county-specific population density factor from treated metadata (log and scaling according to currently published maximum effects); DF is the droplet factor if virus is transmitted by means more highly contagious than droplets, such as aerosols, nuclei, and/or surface contact, the latter two allowing transmission to distances that an air plume can travel before significant dispersion, which is exhaustively studied every day by air pollution engineers; AQF is the Air Quality Factor (either “low” or “not low” for PM and ozone, possibly SOₓ and others as found); VF is the wind velocity factor; MF is the meteorology factor (“low”, “medium” or “high”) based on temperature and humidity; OF is the Other Factor which can be assigned a value based on a short questionnaire and additional metadata as found to relate to distancing or the other safety actions, such as additional susceptibility and transport factors including season, latitude, and atmospheric anomalies, all learned from the COVID-19 and hopefully ready to test more rapidly for the future pandemics; CF is Correction Factor to keep the score scaling between the maximum and minimum when known updates to empirical conditions are established; and w_{iF} are weighting factors used in the source coding to adjust as technology proceeds (Bashir et al 2020, Coccia et al 2020).

If wind roses and lower significant exposure times warrant a higher score for turbulent wind for a particular novel pathogen, then the turbulent wind velocity range will result in a higher score, correlating with the actual velocities predicted in the meteorological forecast metadata. For example, wind roses can show relative wind stability and portion of time in different directions for various wind velocity ranges. So if the wind is predicted to range from 20 to 30 miles per hours and the pathogen has a low significant exposure time, a higher social distance would be warranted especially at ideal transmissibility conditions. Humidity and temperature have been shown to be the most significant meteorological factors for survival of viable respiratory particles. Pneumonia has been shown to be particularly sensitive to weather conditions. Tuberculous has been shown to be transmissible via aerosols resulting in lower significant exposure times needed for infection. If a virus can be transmitted via nuclei, the longest recommended social distances would be recommended with wind transport a major factor, and little to no dependence on temperature and humidity (because evaporation does not render it untransmissible). FIG. 1 shows an example of a wind tunnel effect of a building able to transport respiratory expulsions long distances.

The current example application has been developed for transmission of COVID-19. All weights are currently set to 1.0 and factors ranked according to known influence on transport of respiratory droplets. Dmin=6 feet, Dmax=27 feet. DF=1.0 since COVID-19 is known to transfer via droplets, otherwise it would be much higher for nuclei transmission and a recommendation for other activities, such as avoiding petting others' animals, would accompany. With temperature, humidity and velocity the prime determinants of droplet transport, their maximum values each contributing potentially 40% to the distancing score. With SF, DF, and CF being 1.0, this leaves AQF and OF to contribute a total of 10% each to the score. For each factor, the best- and worst-case range of conditions set their lowest (1.0) and highest values (determined by relative percentage currently as stated). The middle value will be the average of the highest and 1.0.

One of the leading candidates to add/examine in the OF placeholder is the level of atmospheric stability for three land types resulting in different air behavior: coastal, inversion-causing such as in valleys, and in “hinterland” which is translated to mean between coastal and “hinterland.” Hinterland is a concept without direct translation, and a term defined in Google as

“Hinterland is a German word meaning “the land behind” (a city, a port, or similar). Its use in English was first documented by the geographer George Chisholm in his Handbook of Commercial Geography (1888).”

This study found a significant ranking where coastal was found to be safest or requiring the minimum recommended social distancing, hinterland the worst requiring a maximum score for social distancing, and inland valley an average of the two (Coccia et al 2020).

The social distance need due to air velocity, velocity factor, or VF, is most significant and will be scored according to best air regulation engineering guidelines. Since wind velocity is directly proportional to the transport speed of respiratory particles up to the recommended physical distance, at which point they begin to settle, a generally accepted air modelling value of 10 mph for the highest wind speed that is still laminar flow, or in other words without dilution due to eddy diffusion. If the wind speed is 11 mph or greater, the VF is also in highest category in case the user plans to attend populated event or escape/travel route with people in all directions. If future pandemics show longer exposure times needed to contract the illness (i.e. significant exposure time), turbulent flow wind may score in the medium category. Since 10 mph is the worst case, this condition will result in a maximum social distance score no matter what the other conditions are. Since the scale from minimum of 6 feet to maximum of 27 feet is 4.5 (27 feet/6 feet) and indoor air ventilation speed, under which condition the 6-foot-rule was established, is about 7.5 times lower than worst case of 10 mph, a wind speed of zero to 1 mph will result in VF=1.0, a wind speed of 2 mph-9 mph will result in VF=wind speed/2, and a wind speed of 10 mph or more will result in maximum overall score of Equation (1) in this case, currently, of 27 feet regardless of other conditions.

As the modelling develops and evolves, i.e. as neural network paths are enforced, additional “other factors” can be examined and this factor may be split or redistributed with others. The point is to rank factors within primary, secondary and tertiary or assume them equal (or insignificantly different). The factors are then scaled between minimum and maximum social distancing or safety action for that hazard, and split into categories based on empirical knowledge for that hazard. The score will fall into one of those categories and result in a categorical safety action or social distance range, minimum recommendation, etc. However, testing will show how weak or strong additional variables will render the result, with the lesson of less and more reliant is better than too many, resulting in difficult to test. Therefore, the strategy is add factors in cautions stages.

Since factors shown to increase susceptibility and immunity are countless and changing every day, and to be more transparent, Susceptibility Factor may be indicated separately so the user can judge their own additional distancing and compare to the general recommendation for their own information, as is done in EPA's AirNow with increasingly longer forecasting times with the help of Artificial Neural Networks, the exact plan to determine weights if this invention becomes widespread enough to get sufficient data for machine learning. This leaves the distancing score from Eq. (1) a function of factors shown to directly influence environmental transport and all freely available as spatiotemporal forecasting metadata: meteorology, transport particle size, air quality, and other factors shown to directly govern transport as a lumped co-factor with little to no weight since the covariable dependence is still in early stages. Likewise, with case numbers and growth in cases from small pilot studies compared between those who use and do not use this software application, the Other Factor and corresponding weight w_{OF} can be assessed in future calamities and will be adapted as studies go one and are verified, challenged, etc.

Population Factor PF is initially not used until further investigation. While there is support in studies that population density correlated with the COVID-19 outbreak number of cases and speed of that growth, the mechanism by which this was affected remains unclear, but it can serve as a place holder and testing variable for feedback and machine learning of this model, like the other more unsure co-factors increasingly available for automated learning in the universally interdisciplinary metaverse (Martins-Filho, 2021). Therefore, PF will be used in a separate warning assessment along with currently published co-factors freely available as spatiotemporal metadata including socioeconomic area, general regulatory compliance (for example, seatbelt usage by county), breastfeeding rate, per cent free lunches, crime rate, etc.

Air Quality Factor AQF was found to influence transport with other co-factors controlled, and was explained in theory that the pollution can carry the virus further. If the Air Qualify Index is green or yellow, AQF=1.0, if orange or red, AQF=1.2, if purple or maroon, AQF=2.0 (Coumanian et al 2020).

Meteorology Factor MF will initially be either a maximum or minimum value (1.0). Values for most and least transmittable RH and T can be found in the literature which is ever-evolving. For example, if RH is above 50% and Temperature is 33-55 degrees F., this score would be at a maximum, if RH is 50% or less and temperature is above 55 degrees F., this score would be at a minimum, and for all other RH and T this score would be at the average of the maximum and minimum.

The selected factors were shown in literature to have effect on transmissibility and were examined to ensure the contribution ranking did not double count any contribution to the score. The weights were ranked by confidence in contribution to transmissibility and by their relative effect in doubling rates in the early stages of COVID-19 outbreak. For example, all factors relating directly to mass transport, i.e. wind speed, RH and temperature will initially account for at least 68% or one standard deviation of the entire score. Initially they will actually be tested to account for 100% and different increments down to 68% with the factors with the next highest confidence and expected contribution comprising the rest of the score. Factors relating to air stability will be assigned as secondary factors and will initially comprise about 22% of the score, and the rest of the factors will be considered indirect for now, warranting more investigation, so they will have only about 10% of the score and probably tested on at a time since the data volume and complexity will not be trivial. However, initially focusing on meteorological transport factors and gradually adding more has already been accomplished in the app. For now, each factor within the three rankings will have equal weight except for velocity factor, which is more significant than meteorological factor. The top contributors will be tested first.

After the score is generated, increased user susceptibility is factored in and the score is categorized for a tier of social distances or other safety actions. The user susceptibility will be based on emerging literature and estimated to be determined as the technology governing immunology and factors thereof, such as gut metagenomics, is expected to soon unite several medical models (Ericsson 2024).

Finally, the score is scaled to minimum recommended social distance or other safety action, to a maximum (for example, how far can the droplets ever travel before settling or dissipating) or significantly transport elsewise (Bourouiba 2020).

For location and forecasted times, the score from Equation will result in a low, medium and high-risk social distance index, each with their own distancing and safety recommendations. If Equation (1) results in 20 feet or more, the social distancing index will be “red” and indicate that a minimum distancing of 20-30 feet or more for susceptible individuals is recommended. If Equation (1) results in 6-10 feet, the social distancing index will be “green” and indicate that the six-foot rule is predicted to suffice and that 10 feet may be used for more susceptible individuals or for those desiring extra precautions. The other range would be “yellow” and recommend 10 feet to 20 feet, with similar caveats keeping in mind the end result of staying safe and eventual machine learning to improve Equation (1) are only as good as the quality of data, which depends on the user adhering to the recommended distances (or other safety actions and quantities).

Similar embodiments are to be developed for other hazards ranging from wildfire to illegal immigrant detection via remote sensing, to just about any outdoor environmental hazard. For example, the current “super fog” incident in Louisiana leaving drivers suddenly with less than ten feet visibility would be ideal metadata to incorporate from the National Oceanic and Atmospheric Administration (NOAA), as drivers would need real-time predictions of the best route to avoid the super fog, especially for emergency workers.

Conditions can be tested to design partially outdoor event spaces with known safety conditions. For example, the distancing or bacterial destruction can be tested in design of a café that has a garage door in front with fire table just outside, with filtered air entering from the back, exiting the front. This configuration is based on an actual café and laminar flow hoods used to preserve sterile conditions, which are sometimes combined with Bunsen burners for additional contaminant destruction. This same configuration is used in the fields of India for sterile micropropagation of agricultural crops using simple cardboard boxes and candles, and can be used along with the predictions of this invention for additional safety in developing regions.

Predictive modelling metadata input variables of temperature, relative humidity, and wind speed carry so much weight because they are the primary determinants of the environmental transport which determines recommended physical distancing. The range of these values for the hours, days, and portions thereof of the forecast will establish values for these variables based on worst case scenarios for those timeframes, for which the user can click the hyperlink and expansions of forecasts similar to a weather forecast.

Currently the modelling uses only meteorological and locational metadata but will include as warranted the user- and location-specific variables. If triggered, the Droplet Factor can extend the recommended maximum distance to beyond its establish range, possibly resulting in a sheltering recommended action. One example is if the novel pathogen is found to be transmitted via dry particles into the respiratory tract, with non-dispersed plumes of such contaminated air travelling greater distances than can droplets due to settling or aerosols due to evaporation.

This social distance is then categorized into ranges of “low,” “medium” and “high” similar to an Air Quality Forecast or Smoke Forecast, or beach closing which has recently turned into a spatiotemporal prediction map, or forecast (Barkjohn 2022, EPA 2023). This would trigger a green, yellow, or red flag for the user and corresponds to a range of recommended social distancing: at least 6 feet, at least 6-10 feet, and at least 10-27 feet or sheltering recommended. Other accompanying actions are to be included to quickly mitigate effects of transmission (ECDC 2020).

With the uncertainty of these variables, a range of the product of them, each multiplied by a simple starting set of known relative weighting factors, was set at 1.0 for the best conditions resulting in a six-foot physical distance, up to 27.0 feet known maximum for primarily droplet driven transmission.

### Other Technologies to Incorporate

Along with the other environmental media modelling discussed (including groundwater, soil water, surface water, waste water treatment, solid waste, and beyond earth system such as biodomes, biospheres, space habitation, animal habitats etc.), there are models already developed to incorporate with this product including the beach E. coli forecast mapper, ground water infiltration/transport/kinetics including complex systems such as karst, and blackout information for an energy security component of the final software package. Additional environmental security components include food and water security, and of course weather security as this invention is a weather app at the core. The energy and other components have all been exhaustively engineered by public entities so they are the perfect economical social justice collaboration needed as sited by multiple governing entities (EPA, Office of the President, etc.) and their use may actually improve transparency of the methods used to grant competitiveness and therefore survival to industry!

### Summary and Conclusions: Technology Accelerates; Tools are Everything

Technology accelerates. Everything and everyone change every day. Even the relatively recent OSHA and CDC links to pandemic-related definitions such as close contact, and social distancing science are no longer working, this empirical equation is to be used to adapt more theoretical ones used in industry and industrial regulations for our most devastating potential future threats, especially more complex ones such as biohazards and disease.

One main relationship to update with increased understanding and feedback from this software application with modelling is to translate between environmental regulatory equations for pollutant releases and transport of biohazards through the same environmental media.

For example, the AERSCREEN considers land type, building downwash, and release height and direction. Constants exist for different scenarios seen in industrial regulations, and similar ones can be developed for human release without much alteration. Knowing the maximum distance respiratory particles can travel can be used to test different assumptions to translate to biohazards released in various ways.

While the current focus is to prepare the world for the next pandemic from novel pathogens, this technology can be applied with similar methodology for the other environmental media and hazards, especially novel ones with minimal transport, transmission, and exposure characterization.

### REFERENCES

Adeleke O, Akinlabi S A, Jen T-C, Dunmade I. 2021. Application of artificial neural networks for predicting the physical composition of municipal solid waste: An assessment of the impact of seasonal variation. Waste Management & Research. 2021; 39 (8):1058-1068. doi: 10.1177/0734242X21991642
Alexander M, Unruh L, Koval A, Belanger W. 2020. United States response to the COVID-19 pandemic, January-November 2020. Health Econ Policy Law. 2022January; 17 (1):62-75. doi: 10.1017/S1744133121000116. Epub 2021 Mar. 5. PMID: 33663642; PMCID: PMC8060542.
ANDERSON, JANNA, LEE RAINIE AND EMILY A. VOGELS. 2021. Experts Say the ‘New Normal’ in 2025 Will Be Far More Tech-Driven, Presenting More Big Challenges. PEW RESEARCH CENTER. Feb. 18, 2021.
Arumuru V, Pasa J, Samantaray S S. 1994. Experimental visualization of sneezing and efficacy of face masks and shields. Phys Fluids (1994). 2020 Nov. 1; 32 (11):115129. doi: 10.1063/5.0030101. PMID: 33244217; PMCID: PMC7684680.
Ayuso-Mateos J L, Soriano J B, Ancochea J. 2020. Face mask exemptions, psychiatric patients, and COVID-19. Eur Psychiatry. 2020 Dec. 7; 64(1): e6. doi: 10.1192/j.eurpsy.2020.107. PMID: 33280621; PMCID: PMC7804073.
Balboni E, Filippini T, Rothman K J, Costanzini S, Bellino S, Pezzotti P, Brusaferro S, Ferrari F, Orsini N, Teggi S, Vinceti M. 2023. The influence of meteorological factors on COVID-19 spread in Italy during the first and second wave. Environ Res. 2023 Jul. 1; 228:115796. doi: 10.1016/j.envres.2023.115796. Epub 2023 Apr. 3. PMID: 37019296; PMCID: PMC10069087.
Barkjohn K K, Holder A L, Frederick S G, Clements A L. 2022. Correction and Accuracy of PurpleAir PM2.5 Measurements for Extreme Wildfire Smoke. Sensors (Basel). 2022 Dec. 10; 22 (24):9669. doi: 10.3390/s22249669. PMID: 36560038; PMCID: PMC9784900.US
Bashir M F, Ma B, Bilal, Komal B, Bashir M A, Tan D, Bashir M. 2020. Correlation between climate indicators and COVID-19 pandemic in New York, USA. Sci Total Environ. 2020 Aug. 1; 728:138835. doi: 10.1016/j.scitotenv.2020.138835. Epub 2020 Apr. 20. PMID: 32334162; PMCID: PMC7195034.
Bhattacharya S. “Mask Mouth Syndrome”—An emerging oral health threat during the COVID-19 pandemic. J Family Med Prim Care. 2022. August; 11 (8):4869-4870. doi: 10.4103/jfmpc.jfmpc_198_22. Epub 2022 Aug. 30. PMID: 36353050; PMCID: PMC9638642.
Bourouiba L. Turbulent Gas Clouds and Respiratory Pathogen Emissions: Potential Implications for Reducing Transmission of COVID-19. 2020. JAMA. 2020 May 12; 323 (18):1837-1838. doi: 10.1001/jama.2020.4756. PMID: 32215590.
Carson R T, Carson S L, Dye T K, Mayfield S A, Moyer D C, Yu C A. 2021. COVID-19's U.S. Temperature Response Profile. Environ Resour Econ (Dordr). 2021; 80 (4):675-704. doi: 10.1007/s10640-021-00603-8. Epub 2021 Sep. 20. PMID: 34566260; PMCID: PMC8452123.
Cheng, Z., J. Tan, M. Hdeib, J. Kozak. 2003. TISSUE CHARACTERIZATION BY ULTRASOUND SPECKLE. January 2003. DOI: 10.13031/2013.15001. Conference: 2003, Las Vegas, NV Jul. 27-30, 2003.
Chilton, H. 1952. Performance of natural-draught water-cooling towers. Journal of the Institution of Electrical Engineers: Volume 1952, Issue 10, October 1952, Parts 1 and 2.
Christie Aschwanden. 2020. How ‘Superspreading’ Events Drive Most COVID-19 Spread. As few as 10 percent of infected people may drive a whopping 80 percent of cases in specific types of situations. Scientific American Jun. 23, 2020.
Coccia M. 2020. Factors determining the diffusion of COVID-19 and suggested strategy to prevent future accelerated viral infectivity similar to COVID. Sci Total Environ. 2020 Aug. 10; 729:138474. doi: 10.1016/j.scitotenv.2020.138474. Epub 2020 Apr. 20. PMID: 32498152; PMCID: PMC7169901.
Code of Federal Regulations. 82 FR 12285. 2017. Enforcing the Regulatory Reform Agenda. A Presidential Document by the Executive Office of the President on Mar. 1, 2017. Executive Order 13777. Page 12285-12287. Document Number: 2017-04107.
Comunian S, Dongo D, Milani C, Palestini P. 2020. Air Pollution and Covid-19: The Role of Particulate Matter in the Spread and Increase of Covid-19's Morbidity and Mortality. Int J Environ Res Public Health. 2020 Jun. 22; 17 (12):4487. doi: 10.3390/ijerph17124487. PMID: 32580440; PMCID: PMC7345938.
Congress. 1963. Clean Air Act. Agencies: U.S. Environmental Protection Agency. 42 U.S.C. §§ 7401 et seq. Enacted as: the “Clean Air Act”, on Dec. 17, 1963. Clean Air Act.
DiSalvatore R, Bauer S K, Ahn J E, Jahan K. 2023. Development of a COVID-19 Vulnerability Index (CVI) for the Counties and Residents of New Jersey, USA. Int J Environ Res Public Health. 2023 Jul. 7; 20 (13): 6312. doi: 10.3390/ijerph20136312. PMID: 37444160; PMCID: PMC10341843.
Duvall R M, Hagler G S W, Clements A L, Benedict K, Barkjohn K, Kilaru V, Hanley T, Watkins N, Kaufman A, Kamal A, Reece S, Fransioli P, Gerboles M, Gillerman G, Habre R, Hannigan M, Ning Z, Papapostolou V, Pope R, Quintana P J E, Snyder J L. 2020. Deliberating Performance Targets: Follow-on workshop discussing PM10, NO2, CO, and SO2 air sensor targets. Atmos Environ (1994). 2021; 246:10.1016/j.atmosenv.2020.118099. doi: 10.1016/j.atmosenv.2020.118099. PMID: 33746555; PMCID: PMC7970457. USEPA. 2020. Study Explores Ways to Improve Accuracy of Air Sensors. Published May 6, 2020.
Ericsson, Aaron. 2024. Interviews with Aaron Ericsson, DVM, PhD Veterinary Pathology, Director of University of Missouri Metagenomics Center (MUMC).
ESRI. 2023. To Build a New Pollution Map, the US Turned to Thousands of Volunteers. Public Health. Aug. 31, 2023. (ArcGIS)
European Centre for Disease Prevention and Control (ECDC). 2020. Considerations relating to social distancing measures in response to COVID-19-second update. 23 Mar. 2020.
Executive Office of the President. 2023. Presidential Document. Executive Order. E.O. 14096. Apr. 21, 2023. 88 FR 25251. Pp. 25251-25261. Document Number: 2023-08955. Revitalizing Our Nation's Commitment to Environmental Justice for All.
Feng Y, Marchal T, Sperry T, Yi H. 2020. Influence of wind and relative humidity on the social distancing effectiveness to prevent COVID-19 airborne transmission: A numerical study. J Aerosol Sci. 2020 September;147:105585. doi: 10.1016/j.jaerosci.2020.105585. Epub 2020 May 18. PMID: 32427227; PMCID: PMC7233256.
Feng Y, Marchal T, Sperry T, Yi H. 2020. Influence of wind and relative humidity on the social distancing effectiveness to prevent COVID-19 airborne transmission: A numerical study. 2020. J Aerosol Sci. 2020 September;147:105585. doi: 10.1016/j.jaerosci.2020.105585. Epub 2020 May 18. PMID: 32427227; PMCID: PMC7233256.
Ficetola G F, Rubolini D. 2020. Containment measures limit environmental effects on COVID-19 early outbreak dynamics. Sci Total Environ. 2021 Mar. 20; 761:144432. doi: 10.1016/j.scitotenv.2020.144432. Epub 2020 Dec. 16. PMID: 33360124; PMCID: PMC7744010.
Freckelton Qc I. 2020. COVID-19: Fear, quackery, false representations and the law. Int J Law Psychiatry. 2020 September-October; 72:101611. doi: 10.1016/j.ijlp.2020.101611. Epub 2020 Jul. 10. PMID: 32911444; PMCID: PMC7351412.
Iorgulescu G. 2009. Saliva between normal and pathological. Important factors in determining systemic and oral health. J Med Life. 2009 July-September; 2 (3): 303-7. PMID: 20112475; PMCID: PMC5052503.
Isaacs-Thomas, Bella. 2020. Is 6 feet far enough for social distancing? Here's what science says. PBS Newshour, Science. Updated Aug. 26, 2020. Published on Apr. 28, 2020.
Jüni P, Rothenbühler M, Bobos P, Thorpe K E, da Costa B R, Fisman D N, Slutsky A S, Gesink D. 2020. Impact of climate and public health interventions on the COVID-19 pandemic: a prospective cohort study. CMAJ. 2020 May 25; 192 (21): E566-E573. doi: 10.1503/cmaj.200920. Epub 2020 May 8. PMID: 32385067; PMCID: PMC7259972.
Kaur S, Bherwani H, Gulia S, Vijay R, Kumar R. 2021. Understanding COVID-19 transmission, health impacts and mitigation: timely social distancing is the key. Environ Dev Sustain. 2021; 23 (5): 6681-6697. doi: 10.1007/s10668-020-00884-x. Epub 2020 Jul. 18. PMID: 32837280; PMCID: PMC7368631.
Kimbrough, Sue, S. Krabbe, R. Baldauf, T. Barzyk, M. Brown, S. Brown, C. Croghan, M. Davis, P. Deshmukh, R. Duvall, S. Feinberg, V. Isakov, R. Logan, T. McArthur, AND A. Shields. 2019. The Kansas City Transportation and Local-Scale Air Quality Study (KC-TRAQS): Integration of Low-Cost Sensors and Reference Grade Monitoring in a Complex Metropolitan Area. Part 1: Overview of the Project. Chemosensors. MDPI, Basel, Switzerland, 7 (2):26, (2019).
Kopechek J A. 2020. Increased ambient noise and elevated vocal effort contribute to airborne transmission of COVID-19. J Acoust Soc Am. 2020 November; 148 (5):3255. doi: 10.1121/10.0002640. PMID: 33261418; PMCID: PMC7857506.
Künzli N, McConnell R, Bates D, Bastain T, Hricko A, Lurmann F, Avol E, Gilliland F, Peters J. 2003. Breathless in Los Angeles: the exhausting search for clean air. Am J Public Health. 2003 September; 93 (9):1494-9. doi: 10.2105/ajph.93.9.1494. PMID: 12948969; PMCID: PMC1447999.
Li Q, Guan X, Wu P, Wang X, Zhou L, Tong Y. 2020. Early transmission dynamics in Wuhan, China, of novel coronavirus-infected pneumonia. N Engl J Med. 2020;382:1199-207.
Lolli, S., Chen, Y C., Wang, S H. 2020. Impact of meteorological conditions and air pollution on COVID-19 pandemic transmission in Italy. Sci Rep 10, 16213 (2020).
Martins-Filho P R. 2021. Relationship between population density and COVID-19 incidence and mortality estimates: A county-level analysis. J Infect Public Health. 2021 August; 14 (8):1087-1088. doi: 10.1016/j.jiph.2021.06.018. Epub 2021 Jul. 3. PMID: 34245973; PMCID: PMC8253654.
McDermott C V, Alicic R Z, Harden N, Cox E J, Scanlan J M. 2020. Put a lid on it: are faecal bio-aerosols a route of transmission for SARS-CoV-2? J Hosp Infect. 2020 July; 105 (3):397-398. doi: 10.1016/j.jhin.2020.04.024. Epub 2020 Apr. 18. PMID: 32315667; PMCID: PMC7166010.
Mecenas P, Bastos R T D R M, Vallinoto A C R, Normando D. 2020. Effects of temperature and humidity on the spread of COVID-19: A systematic review. PLOS One. 2020 Sep. 18; 15 (9):e0238339. doi: 10.1371/journal.pone.0238339. PMID: 32946453; PMCID: PMC7500589.
Office of Research and Development (ORD). 2018. National Exposure Research Laboratory Ron Williams, David Nash, Gayle Hagler, and Kristen Benedict, US Environmental Protection Agency, Research Triangle Park, NC 27711; Ian C. MacGregor, Brannon A. Seay, and Mitchell Lawrence, Battelle Columbus, OH 43201-2696; Timothy Dye TD Environmental Services. Petaluma, CA 94952. Peer Review and Supporting Literature Review of Air Sensor Technology Performance Targets. EPA/600/R-18/324. September 2018.
Okos, Martin, Nishant Rao, Sarah Drescher and Jeannie Kozak. 1996. Energy Use in the Food Industry: A Study. Published for the American Council for an Energy Efficient Economy. Presented to ACEEE by Jeannie Kozak at Purdue University, West Lafayette, Indiana.
Pathak E B, Menard J M, Garcia R B, Salemi J L. 2022. Joint Effects of Socioeconomic Position, Race/Ethnicity, and Gender on COVID-19 Mortality among Working-Age Adults in the United States. Int J Environ Res Public Health. 2022 Apr. 30; 19 (9):5479. doi: 10.3390/ijerph19095479. PMID: 35564872; PMCID: PMC9102098.
Peci A, Winter A L, Li Y, Gnaneshan S, Liu J, Mubareka S, Gubbay J B. 2919. Effects of Absolute Humidity, Relative Humidity, Temperature, and Wind Speed on Influenza Activity in Toronto, Ontario, Canada. Appl Environ Microbiol. 2019 Mar. 6; 85 (6):e02426-18. doi: 10.1128/AEM.02426-18. PMID: 30610079; PMCID: PMC6414376.
Prata D N, Rodrigues W, Bermejo P H. 2020. Temperature significantly changes COVID-19 transmission in (sub) tropical cities of Brazil. Sci Total Environ. 2020 Aug. 10; 729:138862. doi: 10.1016/j.scitotenv.2020.138862. Epub 2020 Apr. 25. PMID: 32361443; PMCID: PMC7182516.
Purdue University. 2020. Plenary Panel University and Industry Leadership During the Pandemic and Beyond. Webinar.
Qi H, Xiao S, Shi R, Ward M P, Chen Y, Tu W, Su Q, Wang W, Wang X, Zhang Z. 2020. COVID-19 transmission in Mainland China is associated with temperature and humidity: A time-series analysis. Sci Total Environ. 2020 Aug. 1; 728:138778. doi: 10.1016/j.scitotenv.2020.138778. Epub 2020 Apr. 19. PMID: 32335405; PMCID: PMC7167225.
Rohrbach, R. P., McClure, W. F. 1978. A Production Capacity Conveyor for Small Fruit Sorting: The M-Belt. Transactions of the ASAE. 21 (6): 1092-1095. (doi: 10.13031/2013.35447) Published by the American Society of Agricultural and Biological Engineers, St. Joseph, Michigan.
Ron Williams, Vasu Kilaru, and Kristen Benedict. 2018. Air Sensors 2018: Deliberating Performance Targets. A summary of highlights and outcomes from EPA's Air Sensors 2018 Workshop. EM The Magazine for Environmental Managers. A&WMA. October 2018.
Runkle J D, Sugg M M, Leeper R D, Rao Y, Matthews J L, Rennie J J. 2020. Short-term effects of specific humidity and temperature on COVID-19 morbidity in select US cities. Sci Total Environ. 2020 Oct. 20; 740:140093. doi: 10.1016/j.scitotenv.2020.140093. Epub 2020 Jun. 9. PMID: 32540744; PMCID: PMC7280823.
Sajadi M M, Habibzadeh P, Vintzileos A, Shokouhi S, Miralles-Wilhelm F, Amoroso A. 2020. Temperature, Humidity and Latitude Analysis to Predict Potential Spread and Seasonality for COVID-19. SSRN [Preprint]. 2020 Mar. 9: 3550308. doi: 10.2139/ssrn.3550308. Update in: JAMA Netw Open. 2020 Jun. 1; 3 (6):e2011834. PMID: 32714105; PMCID: PMC7366819.
Sanchez-Lorenzo A, Vaquero-Martínez J, Calbó J, Wild M, Santurtun A, Lopez-Bustins J A, Vaquero J M, Folini D, Antón M. 2020. Did anomalous atmospheric circulation favor the spread of COVID-19 in Europe? Environ Res. 2021 March; 194:110626. doi: 10.1016/j.envres.2020.110626. Epub 2020 Dec. 17. PMID: 33345895; PMCID: PMC7746124.
Sarmadi M, Rahimi S, Evensen D, Kazemi Moghaddam V. 2021. Interaction between meteorological parameters and COVID-19: an ecological study on 406 authorities of the UK. Environ Sci Pollut Res Int. 2021 December; 28 (47):67082-67097. doi: 10.1007/s11356-021-15279-2. Epub 2021 Jul. 9. PMID: 34244943; PMCID: PMC8270239.
SAS Institute. 1987. Data Management Procedures for Large and Small Projects. SAS Support. SAS Applications Guide, 1987. Edition, Cary, NC: SAS Institute Inc. SAS Institute Inc. (1985), SAS User's Guide . . . Research Triangle Park, NC 27709-3979.
Shafaati M, Chopra H, Priyanka, Khandia R, Choudhary O P, Rodriguez-Morales A J. 2023. The next pandemic catastrophe: can we avert the inevitable? New Microbes New Infect. 2023 March; 52:101110. doi: 10.1016/j.nmni.2023.101110. Epub 2023 Mar. 10. PMID: 36937540; PMCID: PMC9998279.
Shereen M A, Khan S, Kazmi A, Bashir N, Siddique R. COVID-19 infection: 2020. Origin, transmission, and characteristics of human coronaviruses. 2020. J Adv Res. 2020 Mar. 16; 24:91-98. doi: 10.1016/j.jare.2020.03.005. PMID: 32257431; PMCID: PMC7113610.
TCEQ. 2019. Air Quality Modeling Guidelines. APDG 6232. Air Permits Division. Texas Commission on Environmental Quality. November 2019 Screen 3 and modelling guidance.
USEPA. 2018 (1) Fenceline Monitoring. Finding air pollution leaks with smaller, faster, cheaper measurement technologies. Science Matters. Published May 11, 2018.
USEPA. 2018 (2) Science Matters. Tracking Emissions Using New Fenceline Monitoring Technology. Published Jun. 18, 2018
USEPA. 2018 (3) Conversations with Ann Brown EPA regarding interpretation and implementation of the Fenceline Monitoring Program.
USEPA. 2020 (1) Electronic Code of Federal Regulations (e-CFR) Title 40—Protection of Environment CHAPTER I—ENVIRONMENTAL PROTECTION AGENCY SUBCHAPTER C—AIR PROGRAMS PART 63—NATIONAL EMISSION STANDARDS FOR HAZARDOUS AIR POLLUTANTS FOR SOURCE CATEGORIES Subpart CC—National Emission Standards for Hazardous Air Pollutants From Petroleum Refineries § 63.658 Fenceline monitoring provisions. Appendix A. Methods 325A and 325B.
USEPA. 2020 (2) 40 CFR Part 63. [EPA-HQ-OAR-2010-0682; FRL 10004-55-OAR] RIN 2016-AT18. National Emission Standards for Hazardous Air Pollutants: Petroleum Refinery Sector. Federal Register. Vol. 85, No. 23. Tuesday, Feb. 4, 2020. Rules and Regulations. Pp 6064-6087.
USEPA. 2020 (3) Science Matters. DIY Air Sensor Now Available for Use by Educators and Citizen Scientist. Published Sep. 14, 2020.
USEPA. 2021. USEPA Citizen Science Community and Citizen Science Webinar Archive. This webinar, recorded on Dec. 15, 2021, showcased community-oriented projects and ideas from a recent workshop on community and citizen science. The workshop explored gaps and needed improvements in data management that will allow for a more efficient flow of data from producers to users. The webinar included topics on how we can maximize use of community and citizen science data, addressing barriers that limit data use, and how the EPA can better serve as a catalyst to build a more inclusive and collaborative network with states, tribes, local government, NGOs, academia and other organizations. The webinar also highlighted several case studies of citizen science projects including Love My Air Denver, Atlanta Urban Heat, the Eastern Shale Gas Monitoring Program, and Arizona Water Watch.
USEPA. 2023 (1) EPA Air Sensor Toolbox. Evaluation of Emerging Air Sensor Performance.
USEPA. 2023. (2) Science Matters. EPA Research Updates. Is your job beach? Oct. 4, 2023.
Wang C C, Prather K A, Sznitman J, Jimenez J L, Lakdawala S S, Tufekci Z, Marr L C. Airborne transmission of respiratory viruses. 2021. Science. 2021 Aug. 27; 373 (6558):eabd9149. doi:10.1126/science.abd9149. PMID: 34446582; PMCID: PMC8721651.
Williams S B, Shan Y, Jazzar U, Kerr P S, Okereke I, Klimberg V S, Tyler D S, Putluri N, Lopez D S, Prochaska J D, Elferink C, Baillargeon J G, Kuo Y F, Mehta H B. 2020. Proximity to Oil Refineries and Risk of Cancer: A Population-Based Analysis. JNCI Cancer Spectr. 2020 Oct. 7; 4 (6):pkaa088. doi: 10.1093/jncics/pkaa088. PMID: 33269338; PMCID: PMC7691047.
Williams, R. R. Duvall, V. Kilaru, G. Hagler, L. Hassinger, K. Benedict, J. Rice, A. Kaufman, R. Judge, G. Pierce, G. Allen, M. Bergin, R. C. Cohen, P. Fransioli, M. Gerboles, R. Habre, M. Hannigan, D. Jack, P. Louie, N. A. Martin, M. Penza, A. Polidori, R. Subramanian, K. Ray, J. Schauer, E. Seto, G. Thurston, J. Turner, A. S. Wexler, Z. Ning. 2019. Deliberating performance targets workshop: Potential paths for emerging PM2.5 and O3 air sensor progress. Atmospheric Environment: X, Volume 2, 2019, 100031, ISSN 2590-1621.
Wu Y, Jing W, Liu J, Ma Q, Yuan J, Wang Y, Du M, Liu M. 2020. Effects of temperature and humidity on the daily new cases and new deaths of COVID-19 in 166 countries. Sci Total Environ. 2020 Aug. 10; 729:139051. doi: 10.1016/j.scitotenv.2020.139051. Epub 2020 Apr. 28. PMID: 32361460; PMCID: PMC7187824.
Zhao L, Qi Y, Luzzatto-Fegiz P, Cui Y, Zhu Y. 2020. COVID-19: Effects of Environmental Conditions on the Propagation of Respiratory Droplets. Nano Lett. 2020 Oct. 14; 20 (10):7744-7750. doi: 10.1021/acs.nanolett.Oc03331. Epub 2020 Sep. 15. PMID: 32909761.

1. Predictive modelling has been demonstrated in a software application for mobile or personal computer which forecasts site-specific, user-specific recommendations for different levels of physical distancing from novel airborne pathogen sources, and it can also forecast corrective and/or safety actions to mitigate other environmental hazards, and be combined with other, existing environmental predictive safety models for an economic full safety software package, with the recommended corrective actions including social distances, safety actions, and/or control of the source, receptor, or path in between, with early safety actions and distancing found to be key to preventing future mass death from novel pandemics (Kaur 2021), the main initial calculation with the highest degree of accuracy will be social distancing and appropriate actions to maintain survival from the early pandemic identification and preliminary transmissibility characterization studies, because the main use is outdoor calculation of safety actions since the modelling currently relies on meteorological data, but this invention with modelling would likewise predict mitigation actions and quantities anytime the air or other environmental/ambient media behaves similarly.
